(19) United States
(12) Patent Application Publication
(10) Pub. No.: US 2005/0282824 A1
(43) Pub. Date: Dec. 22, 2005

(54) Substituted pyrimidines as inhibitors of bacterial type III protein secretion systems

(76) Inventor: Xiaobing Li, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/124,226

(22) Filed: May 6, 2005

Related U.S. Application Data
(60) Provisional application No. 60/568,850, filed on May 7, 2004.

(51) Int. Cl. C07D 43/02; A61K 31/506

(52) U.S. Cl. 514/256; 544/328; 544/295; 514/252.14

(57) ABSTRACT

In accordance with the present invention, compounds that inhibit Type III protein section have been identified, and methods for their use provided. In one aspect of the invention, compounds useful in the inhibition of Type III protein section and/or in the treatment and prevention of bacterial infections, particularly Gram-negative bacterial infections, are provided. In another aspect of the invention, methods are provided for the inhibition of Type III protein secretion and/or the treatment and prevention of bacterial infections, particularly Gram-negative bacterial infections using the compounds of the invention.

### CROSS-REFERENCE TO RELATED APPLICATION

This applications claims the benefit under 35 U.S.C. 119(e) of provisional application, Ser. No. 60/568,850, filed May 7, 2004.

### FIELD OF THE INVENTION

The subject invention relates to novel substituted pyrimidines that have anti-microbial properties, their compositions and their uses.

### BACKGROUND OF THE INVENTION

Type III protein secretion systems are an essential virulence determinant of most pathogenic Gram-negative bacteria, including Salmonella, Shigella, Yersinia, Pseudomonas aeruginosa, and enteropathogenic Escherichia coli. The Type III virulence mechanism consists of a secretion apparatus, consisting of about 25 proteins, and a set of effector proteins released by this apparatus. Following activation by intimate contact with a eukaryotic cell membrane, the effector proteins are injected into the host cell, where they subvert the signal transduction machinery and lead to a variety of host cell responses. This virulence mechanism plays a key role in establishing and maintaining an infection and in the resulting pathophysiological sequelae, such as diarrhea, chronic lung inflammation, and septicemia.

Certain protein components of the Type III secretion apparatus are highly conserved among bacterial pathogens, and as such represent suitable targets for therapeutic intervention. Inhibitors of Type III protein secretion are expected to be useful as prophylactic agents (i.e., to prevent the onset of infection by Gram-negative bacteria) or as drugs to treat an existing bacterial infection, either with or without an anti-bacterial agent.

There remains a need to develop, characterize, and optimize lead molecules for the development of novel anti-bacterial drugs. Accordingly, it is an object of the present invention to provide such compounds.

### SUMMARY OF THE INVENTION

In accordance with the present invention, compounds that inhibit Type III protein secretion have been identified, and methods for their use provided.

In one aspect of the invention, compounds of Formula (I) are provided which are useful in the inhibition of Type III protein secretion and/or in the treatment and prevention of bacterial infection, particularly Gram-negative bacterial infection.

In another aspect of the invention, methods are provided for the inhibition of Type III protein secretion and/or in the treatment and prevention of bacterial infection, particularly Gram-negative bacterial infection using the compounds described herein.

In one embodiment, the invention is directed to methods for inhibiting Type III protein secretion comprising administering a secretion-inhibiting amount of at least one compound of the invention to a subject in need thereof.

In another embodiment, methods for treating and/or preventing bacterial infection, particularly Gram-negative bacterial infection, are provided comprising administering a therapeutically or prophylactically effective amount of at least one compound of the invention to a subject in need thereof.

These and other aspects of the invention will be more clearly understood with reference to the following preferred embodiments and detailed description.

### DETAILED DESCRIPTION OF THE INVENTION

Inhibition of Type III protein secretion is an important factor in the treatment and prevention of bacterial infection. In accordance with the present invention, compounds that inhibit Type III protein secretion have been identified, and methods for their use provided.

A. Compounds of the Invention

In one aspect of the invention, compounds of the invention are provided which are useful in the inhibition of bacterial Type III protein secretion systems, and/or in the treatment or prevention of bacterial infection, particularly Gram-negative bacterial infection.

Where the compounds according to this invention have at least one stereogenic center, they may accordingly exist as enantiomers. Where the compounds possess two or more stereogenic centers, they may additionally exist as diastereomers. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Some of the compounds of the present invention may have trans and cis isomers. In addition, where the processes for the preparation of the compounds according to the invention give rise to a mixture of stereoisomers, these isomers may be separated by conventional techniques such as preparative chromatography. The compounds may be prepared as a single stereoisomer or in racemic form as a mixture of some possible stereoisomers. The non-racemic forms may be obtained by either synthesis or resolution. The compounds may, for example, be resolved into their component enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography.

Certain of the compounds of the invention, for example the imidazole derivatives, may exist as tautomers. It is understood that such tautomeric forms are intended to be encompassed within the scope of the invention.

As used herein, “enantiomerically pure” refers to compositions consisting substantially of a single isomer, preferably consisting of 90%, 92%, 95%, 98%, 99%, or 100% of a single isomer.

Included within the scope of the invention are the hydrated forms of the compounds that contain various amounts of water, for instance, the hydrate, hemihydrate, and sesquihydrate forms. The present invention also includes within its scope prodrugs and pharmaceutically acceptable salts of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

Preferred compounds of the present invention useful in the inhibition of Type III protein secretion include those of Formula (I) as shown below.

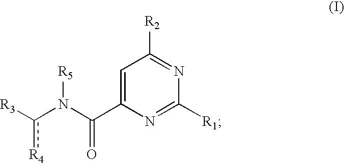

wherein R₁ is halogen; aryl, substituted aryl, heteroaryl, or heterocyclyl, optionally substituted by one or more lower alkyl, aryl or heterocyclyl;

R₂ is lower alkyl, aryl, substituted aryl, heteroaryl, heterocyclyl, or substituted heterocyclyl;

R₃ is hydrogen or carboxy;

R₄ is lower alkyl, optionally substituted by aryl, substituted aryl, benzyloxy, or benzylthio; or methylene;

R₅ is hydrogen or lower alkyl;

or an optical isomer, diastereomer or enantiomer thereof, or a pharmaceutically acceptable salt, hydrate, ester or prodrug thereof.

More particularly preferred compounds of the present invention useful in the inhibition of Type III protein secretion include:

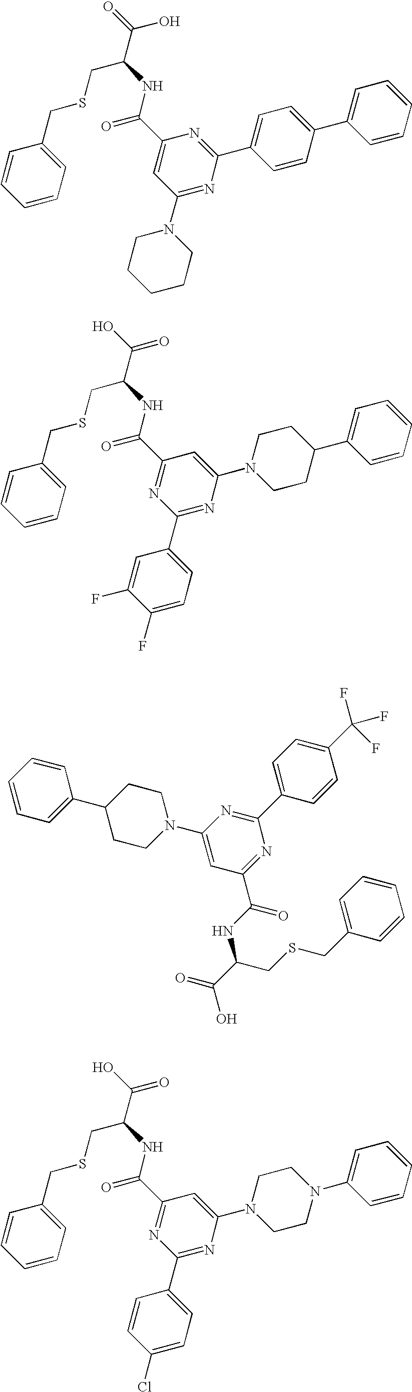

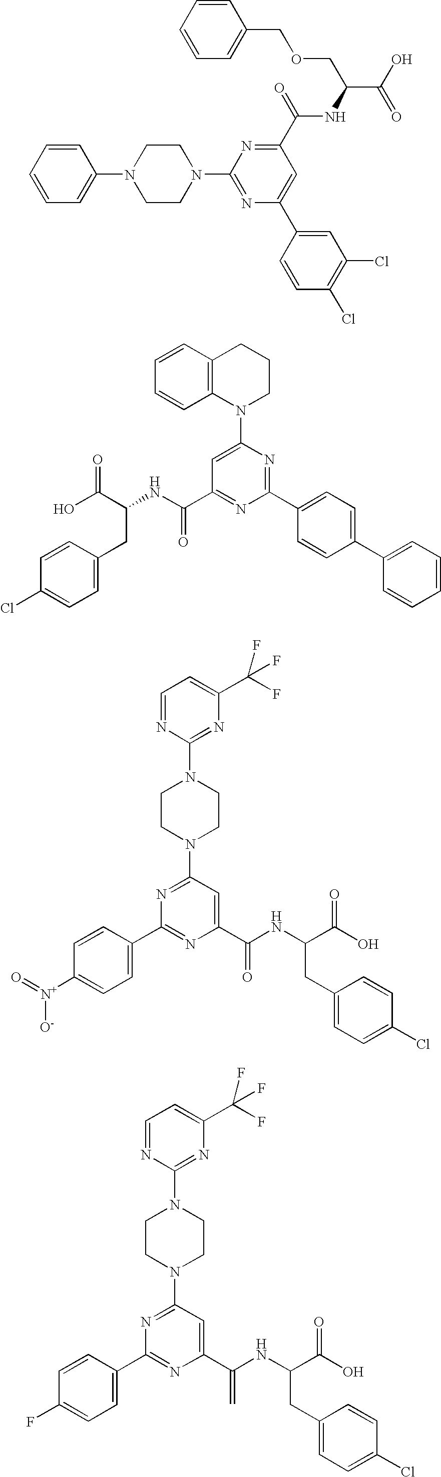

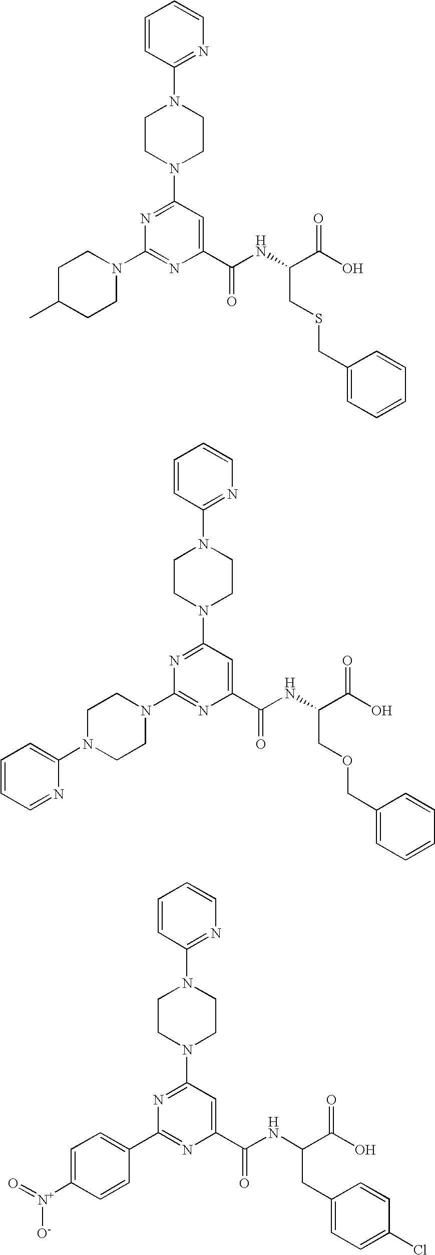

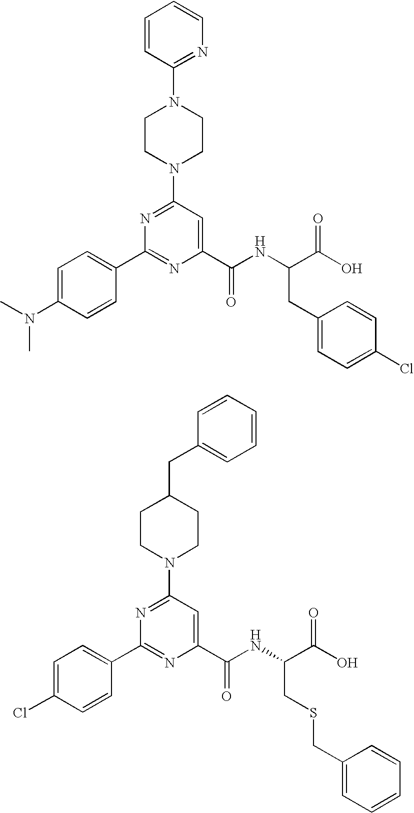

Relative to the above description, certain definitions apply as follows.

Unless otherwise noted, under standard nomenclature used throughout this disclosure the terminal portion of the designated side chain is described first, followed by the adjacent functionality toward the point of attachment.

Unless specified otherwise, the terms “alkyl,” “alkenyl,” and “alkynyl,” whether used alone or as part of a substituent group, include straight and branched chains having 1 to 8 carbon atoms, or any number within this range. The term “alkyl” refers to straight or branched chain hydrocarbons. “Alkenyl” refers to a straight or branched chain hydrocarbon with at least one carbon-carbon double bond. “Alkynyl” refers to a straight or branched chain hydrocarbon with at least one carbon-carbon triple bond. For example, alkyl radicals include methyl, ethyl, n-propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl.

“Alkoxy” radicals are oxygen ethers formed from the previously described straight or branched chain alkyl groups.

“Cycloalkyl” groups contain 3 to 8 ring carbons and preferably 5 to 7 ring carbons.

The alkyl, alkenyl, alkynyl, cycloalkyl groups and alkoxy groups may be independently substituted with one or more members of the group including, but not limited to, halogen, alkyl, alkenyl, alkynyl, cycloalkyl, oxo, aryl, heteroaryl, heterocyclo, CN, nitro, —OCOR₅, —OR₅, —SR₅, —SOR₅, —SO₂R₅, —COOR₅, —NR₅R₆, —CONR₅R₅R₆, —OCONR₅R₆, —NHCOR₅, —NHCOOR₅, —NHC(NH)NHNO₂, and —NHCONR₅R₆, wherein R₅ and R₆ are independently selected from hydrogen, alkyl, alkenyl, alkynyl, cycloalkyl, aryl, heteroaryl, heterocyclo, aralkyl, heteroaralkyl, and heterocycloalkyl, or alternatively R₅ and R₆ may join to form a heterocyclic ring containing the nitrogen atom to which they are attached.

The term “acyl” as used herein, whether used alone or as part of a substituent group, means an organic radical having 2 to 6 carbon atoms (branched or straight chain) derived from an organic acid by removal of the hydroxyl group. The term “Ac” as used herein, whether used alone or as part of a substituent group, means acetyl.

The term “halo” or “halogen” means fluoro, chloro, bromo or iodo. (Mono-, di-, tri-, and per-)halo-alkyl is an alkyl radical substituted by independent replacement of the hydrogen atoms thereon with halogen.

“Aryl” or “Ar,” whether used alone or as part of a substituent group, is a carbocyclic aromatic radical including, but not limited to, phenyl, 1- or 2-naphthyl and the like. The carbocyclic aromatic radical may be substituted by independent replacement of 1 to 3 of the hydrogen atoms thereon with aryl, heteroaryl, halogen, OH, CN, mercapto, nitro, amino, C₁-C₈-alkyl, C₂-C₈-alkenyl, C₁-C₈-alkoxy, C₁-C₈-alkylthio, C₁-C₈-alkyl-amino, di(C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, or carboxamide. Illustrative aryl radicals include, for example, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimethylcarbamylphenyl and the like. “Ph” or “PH” denotes phenyl. “Bz” denotes benzoyl.

Whether used alone or as part of a substituent group, “heteroaryl” refers to a cyclic, fully unsaturated radical having from five to ten ring atoms of which one ring atom is selected from S, O, and N; 0-2 ring atoms are additional heteroatoms independently selected from S, O, and N; and the remaining ring atoms are carbon. The radical may be joined to the rest of the molecule via any of the ring atoms. Exemplary heteroaryl groups include, for example, pyridinyl, pyrazinyl, pyrimidinyl, pyridazinyl, pyrrolyl, pyrazolyl, imidazolyl, thiazolyl, oxazolyl, isoxazolyl, thiadiazolyl, triazolyl, triazinyl, oxadiazolyl, thienyl, furanyl, quinolinyl, isoquinolinyl, indolyl, isothiazolyl, N-oxo-pyridyl, 1,1-dioxothienyl, benzothiazolyl, benzoxazolyl, benzothienyl, quinolinyl-N-oxide, benzimidazolyl, benzisothiazolyl, benzisoxazolyl, benzodiazinyl, benzofurazanyl, indazolyl, indolizinyl, benzofuryl, cinnolinyl, quinoxalinyl, pyrrolopyridinyl, furopyridinyl (such as furo[2,3-c]pyridinyl, furo[3,2-b]pyridinyl, or furo[2,3-b]pyridinyl), imidazo-pyridinyl (such as imidazo[4,5-b]pyridinyl or imidazo[4,5-cpyridinyl), naphthyridinyl, phthalazinyl, purinyl, pyridopyridyl, quinazolinyl, thienofuryl, thienopyridyl, and thienothienyl. The heteroaryl group may be substituted by independent replacement of 1 to 3 of the hydrogen atoms thereon with aryl, heteroaryl, halogen, OH, CN, mercapto, nitro, amino, C₁-C₈-alkyl, C₁-C₈-alkoxy, C₁-C₈-alkylthio, C₁-C₈-alkyl-amino, di(C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, or carboxamide. Heteroaryl may be substituted with a mono-oxo to give for example a 4-oxo-1H-quinoline.

The terms “heterocycle,” “heterocyclic,” and “heterocyclo” refer to an optionally substituted, fully saturated, partially saturated, or non-aromatic cyclic group which is, for example, a 4- to 7-membered monocyclic, 7- to 11-membered bicyclic, or 10- to 15-membered tricyclic ring system, which has at least one heteroatom in at least one carbon atom containing ring. Each ring of the heterocyclic group containing a heteroatom may have 1, 2, or. 3 heteroatoms selected from nitrogen atoms, oxygen atoms, and sulfur atoms, where the nitrogen and sulfur heteroatoms may also optionally be oxidized. The nitrogen atoms may optionally be quaternized. The heterocyclic group may be attached at any heteroatom or carbon atom. The heterocyclic group may be substituted by independent replacement of 1 to 3 of the hydrogen atoms thereon with aryl, heteroaryl, halogen, C₁-C₈-alkyl, C₁-C₈-alkoxy, carboxy, alkoxycarbonyl, or carboxamide.

Exemplary. monocyclic heterocyclic groups include pyrrolidinyl; oxetanyl; pyrazolinyl; imidazolinyl; imidazolidinyl; oxazolinyl; oxazolidinyl; isoxazolinyl; thiazolidinyl; isothiazolidinyl; tetrahydrofuryl; piperidinyl; piperazinyl; 2-oxopiperazinyl; 2-oxopiperidinyl; 2-oxopyrrolidinyl; 4-piperidonyl; tetrahydropyranyl; tetrahydrothiopyranyl; tetrahydrothiopyranyl sulfone; morpholinyl; thiomorpholinyl; thiomorpholinyl sulfoxide; thiomorpholinyl sulfone; 1,3-dioxolane; dioxanyl; thietanyl; thiiranyl; 2-oxazepinyl; azepinyl; and the like. Exemplary bicyclic heterocyclic groups include quinuclidinyl; tetrahydroisoquinolinyl; dihydroisoindolyl; dihydroquinazolinyl (such as 3,4-dihydro-4-oxo-quinazolinyl); dihydrobenzofuryl; dihydrobenzothienyl; benzothiopyranyl; dihydrobenzothiopyranyl; dihydrobenzothiopyranyl sulfone; benzopyranyl; dihydrobenzopyranyl; indolinyl; chromonyl; coumarinyl; isochromanyl; isoindolinyl; piperonyl; tetrahydroquinolinyl; and the like.

Substituted aryl, substituted heteroaryl, and substituted heterocycle may also be substituted with a second substituted aryl, a second substituted heteroaryl, or a second substituted heterocycle to give, for example, a 4-pyrazol-1-yl-phenyl or 4-pyridin-2-yl-phenyl.

The term “carbocyclic” refers to a saturated or unsaturated, non-aromatic, monocyclic, hydrocarbon ring of 3 to 7 carbon atoms.

Designated numbers of carbon atoms (e.g., C₁-C₈ or C₁₋₈) shall refer independently to the number of carbon atoms in an alkyl or cycloalkyl moiety or to the alkyl portion of a larger substituent in which alkyl appears as its prefix root.

The term “hydroxy protecting group” refers to groups known in the art for such purpose. Commonly used hydroxy protecting groups are disclosed, for example, in T. H. Greene and P. G. M. Wuts, Protective Groups in Organic Synthesis, 2nd edition, John Wiley & Sons, New York (1991), which is incorporated herein by reference. Illustrative hydroxyl protecting groups include but are not limited to tetrahydropyranyl; benzyl; methylthiomethyl; ethythiomethyl; pivaloyl; phenylsulfonyl; triphenylmethyl; trisubstituted silyl such as trimethylsilyl, triethylsilyl, tributylsilyl, tri-isopropylsilyl, t-butyldimethylsilyl, tri-t-butylsilyl, methyldiphenylsilyl, ethyldiphenylsilyl, t-butyldiphenylsilyl; acyl and aroyl such as acetyl, benzoyl, pivaloylbenzoyl, 4-methoxybenzoyl, 4-nitrobenzoyl and phenylacetyl.

The phrase “a pharmaceutically acceptable salt” denotes one or more salts of the free base or free acid which possess the desired pharmacological activity of the free base or free acid as appropriate and which are neither biologically nor otherwise undesirable. These salts may be derived from inorganic or organic acids. Examples of inorganic acids are hydrochloric acid, nitric acid, hydrobromic acid, sulfuric acid, or phosphoric acid. Examples of organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, benzenesulfonic acid, salicylic acid and the like. Suitable salts are furthermore those of inorganic or organic bases, such as KOH, NaOH, Ca(OH)₂, Al(OH)₃, piperidine, morpholine, ethylamine, triethylamine and the like.

The term “subject” includes, without limitation, any animal or artificially modified animal. As a particular embodiment, the subject is a human.

The term “drug-resistant” or “drug-resistance” refers to the characteristics of a microbe to survive, in the presence of a currently available antimicrobial agent such as an antibiotic at its routine, effective concentration.

Unless specified otherwise, it is intended that the definition of any substituent or variable at a particular location in a molecule be independent of its definitions elsewhere in that molecule, It is understood that substituents and substitution patterns on the compounds of this invention can be selected by one of ordinary skill in the art to provide compounds that are chemically stable and that can be readily synthesized by techniques known in the art as well as those methods set forth herein. Further, where a more generic substituent is set forth for any position in the molecules of the present invention, it is understood that the generic substituent may be replaced with more specific substituents, and the resulting molecules are within the scope of the molecules of the present invention.

B. Preparation of Compounds of the Invention

Compounds of the invention may be produced in any manner known in the art. By way of example, compounds of the invention may be prepared according to the following general schemes. The skilled artisan will also recognize the judicious choice of reactions, solvents, and temperatures are an important component in successful synthesis. While the determination of optimal conditions, etc. is routine, it will be understood that a variety of compounds can be generated in a similar fashion, using the guidance of the schemes below.

The starting materials used in preparing the compounds of the invention are known, made by published synthetic methods or available from commercial vendors.

It is recognized that the skilled artisan in the art of organic chemistry can readily carry out standard manipulations of the organic compounds without further direction; that is, it is well within the scope and practice of the skilled artisan to carry out such manipulations. These include, but are not limited to, reductions of carbonyl compounds to their corresponding alcohols, oxidations, acylations, aromatic substitutions, both electrophilic and nucleophilic, etherifications, esterification and saponification and the like. Examples of these manipulations are discussed in standard texts such as March, Advanced Organic Chemistry (Wiley), Carey and Sundberg, Advanced Organic Chemistry (Vol. 2), Feiser & Feiser, Reagents for Organic Synthesis (16 volumes), L. Paquette, Encyclopedia of Reagents for Organic Synthesis (8 volumes), Frost & Fleming, Comprehensive Organic Synthesis (9 volumes) and the like.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. Examples of these manipulations can be found for example in T. Greene, Protecting Groups in Organic Synthesis.

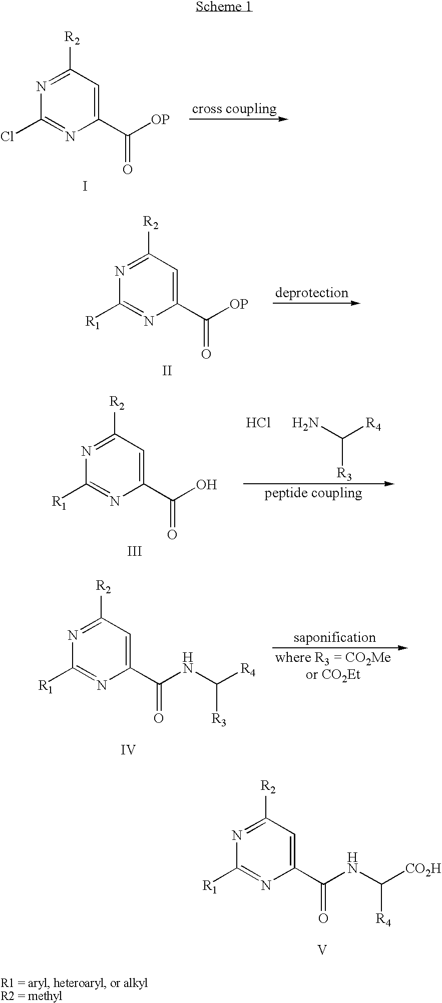

Pyrimidine (V) of formula 1, wherein Q₁ is a suitably substituted aryl, heteroaryl or alkyl group, Q₂ is methyl and Q₃ is hydrogen, can be prepared by the method outlined in Scheme 1. Cross-coupling reaction at the C-2 position of (I) can be achieved by one of the following methods:

1) a palladium-catalyzed Suzuki coupling reaction, with a suitably substituted aryl or heteroaryl boronic acid, catalyzed by, for example, tetrakis(triphenylphosphine)palladium(0) ((PPh₃)₄Pd), bis(dibenzylideneacetone)palladium(0) (Pd(dba)₂), bis(tri-tert-butyl-phosphine)palladium(0) (Pd(P^{t}Bu₃)₂), or dichlorobis(triphenylphosphine)palladium(II) ((PPh₃)₂PdCl₂), using tris(o-furyl)phosphine (TFP), triphenylphosphine (TPP) or 1,1′-bis(diphenylphosphino)fefrocene (dppf) as ligand, in the presence of a base, such as, triethylamine, cesium fluoride, sodium carbonate or sodium tert-butoxide; and

2) a Suzuki-Miyaura coupling reaction of an alkyl 9-BBN derivative catalyzed by, for example, PdCl₂(dppf), in the presence of a suitable base, such as cesium carbonate, sodium hydroxide, or the like.

Typically these reactions are carried out in a suitable solvent, such as tetrahydrofuran, tetrahydrofuran/water mixture, methanol, dimethylformamide (DMF) or dimethoxyethane (DME), for from 1 to 48 hours at a temperature ranging from 0° C. to 80° C. Alternatively, these reactions can be carried out in a microwave in a sealed tube at an elevated reaction temperature, ranging from 80° C. to 160° C. Removal of the ester protecting group of (II), for example by treatment with an acid, such as formic acid or trifluoroacetic acid, in the case of a t-butyl ester derivative, or by saponification with an alkali metal hydroxide, such as lithium hydroxide, sodium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, in the case of a methyl or ethyl ester, provides the corresponding acid derivative (III). The conversion of acid (III) to amide (IV) can be carried out by reaction of (III) with an amine nucleophile, such as an amino acid ester hydrochloride, and a suitable peptide coupling reagent, such as DCC, EDCI, PyBop, PyBrop, HATU, or the like, optionally in the presence of a suitable base, such as triethylamine, diisopropylethylamine, or the like, in an inert solvent, such as dichloromethane, chloroform, or tetrahydrofuran. The reaction is conducted at a temperature from −20° C. to 37° C. for from 2 to 48 hours. In the case where R₃ is an ester functionality, such as CO₂Me or CO₂Et, saponification with an alkali metal hydroxide, such as sodium hydroxide, lithium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, provides the corresponding acid derivative (V).

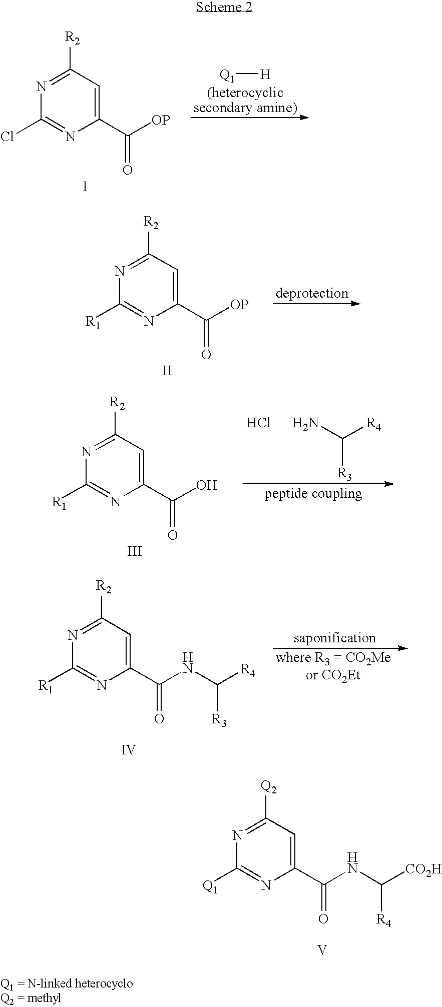

Pyrimidine (V) of formula 1, wherein Q₁ is an N-linked heterocycle, Q₂ is methyl and Q₃ is hydrogen, can be prepared by the method outlined in Scheme 2. Reaction of (I) with a heterocyclic secondary amine, such as an optionally substituted piperidine or piperazine derivative, optionally in the presence of a suitable base, such as triethylamine, diisopropylethylamine, or the like, in an inert solvent, such as dichloromethane, chloroform, or tetrahydrofuran at a temperature ranging from −20° C. to 37° C. for from 1 to 24 hours, provides pyrimidine derivative (II). Removal of the ester protecting group of (II), for example, by treatment with an acid, such as formic acid or trifluoroacetic acid, in the case of a t-butyl ester derivative, or by saponification with an alkali metal hydroxide, such as lithium hydroxide, sodium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, in the case of a methyl or ethyl ester, provides the corresponding acid derivative (III). The conversion of acid (III) to amide (IV) can be carried out by reaction of (III) with an amine nucleophile, such as an amino acid ester hydrochloride, and a suitable peptide coupling reagent, such as DCC, EDCI, PyBop, PyBrop, HATU, or the like, optionally in the presence of a suitable base, such as triethylamine, diisopropylethylamine, or the like, in an inert solvent, such as dichloromethane, chloroform, or tetrahydrofuran. The reaction is conducted at a temperature from −20° C. to 37° C. for from 2 to 48 hours. In the case where R₃ is an ester functionality, such as CO₂Me or CO₂Et, saponification with an alkali metal hydroxide, such as sodium hydroxide, lithium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, provides the corresponding acid derivative (V).

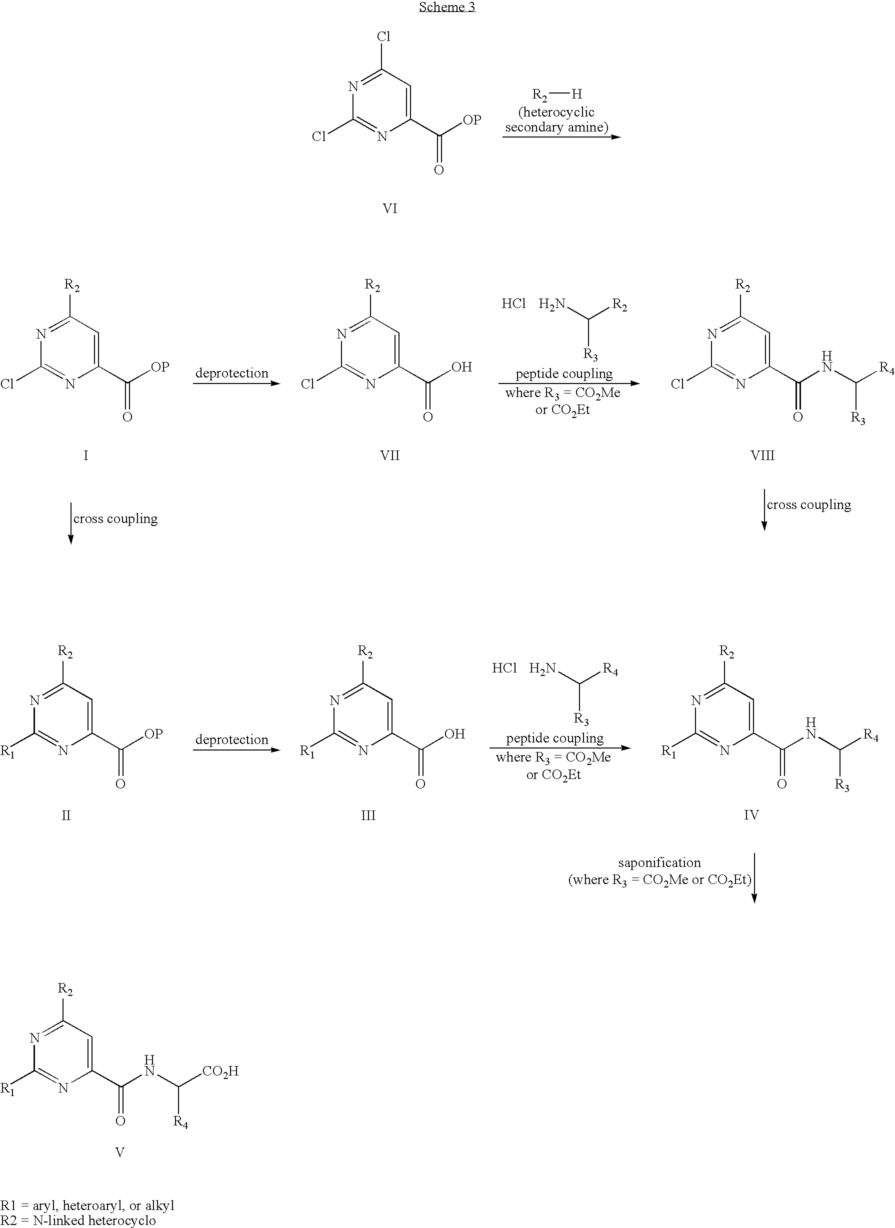

Pyrimidine (V) of formula 1, wherein Q₁ is a suitably substituted aryl, heteroaryl or alkyl group and Q₂ is an N-linked heterocycle, can be prepared by the method outlined in Scheme 3 (MJM: What is Q₃?). Reaction of dichloropyrimidine (VI) with a heterocyclic secondary amine, such as an optionally substituted piperidine or piperazine derivative, optionally in the presence of a suitable base, such as triethylamine, diisopropylethylamine, or the like, in an inert solvent, such as dichloromethane, chloroform, or tetrahydrofuran at a temperature ranging from −20° C. to 37° C. for from 1 to 24 hours, provides pyrimidine derivative (VII). Cross-coupling reaction at the C-2 position of (I) can be achieved by one of the following methods to give compound (II):

1) a palladium-catalyzed Suzuki coupling reaction, with a suitably substituted aryl or heteroaryl boronic acid, catalyzed by, for example, tetrakis(triphenylphosphine)palladium(0) ((PPh₃)₄Pd), bis(di benzylidene-acetone)palladium(0) (Pd(dba)₂), bis(tri-tert-butylphosphine)palladium(0) (Pd(P^{t}Bu₃)₂), or dichlorobis(triphenylphosphine) palladium(II) ((PPh₃)₂PdCl₂), using tris(o-furyl)phosphine (TFP), triphenylphosphine (TPP) or 1,1′-bis(diphenylphosphino)ferrocene (dppf) as ligand, in the presence of a base, such as, triethylamine, cesium fluoride, sodium carbonate or sodium tert-butoxide; and

2) a Suzuki-Miyaura coupling reaction of an alkyl 9-BBN derivative catalyzed by, for example, PdCl₂(dppf), in the presence of a suitable base, such as cesium carbonate, sodium hydroxide, or the like.

Typically, these reactions are carried out in a suitable solvent, such as tetrahydrofuran, tetrahydrofuran/water mixture, methanol, dimethylformamide (DMF) or dimethoxyethane (DME), for from 1 to 48 hours at a temperature ranging from 0° C. to 80° C. Alternatively, these reactions can be conducted in a microwave in a sealed tube at an elevated temperature, ranging from 80° C. to 160° C. Removal of the ester protecting group of (II), for example by treatment with an acid, such as formic acid or trifluoroacetic acid, in the case of a t-butyl ester derivative, or by saponification with an alkali metal hydroxide, such as lithium hydroxide, sodium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, in the case of a methyl or ethyl ester derivative, provides the corresponding acid derivative (III). The conversion of acid (III) to amide (IV) can be carried out by reaction of (III) with an amine nucleophile, such as an amino acid ester hydrochloride, and a suitable peptide coupling reagent, such as DCC, EDCI, PyBop, PyBrop, HATU, or the like, optionally in the presence of a suitable base, such as triethylamine, diisopropylethylamine, or the like, in an inert solvent, such as dichloromethane, chloroform, or tetrahydrofuran. The reaction is conducted at a temperature from −20° C. to 37° C. for from 2 to 48 hours. In the case where R₃ is an ester functionality, such as CO₂Me or CO₂Et, saponification with an alkali metal hydroxide, such as sodium hydroxide, lithium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, provides the corresponding acid derivative (V). It should be apparent to one skilled in the art that the order of steps in the reaction sequence can be altered (i.e., I→VII→VIII→IV→V) to obtain the same compounds of Formula I as shown in Scheme 3.

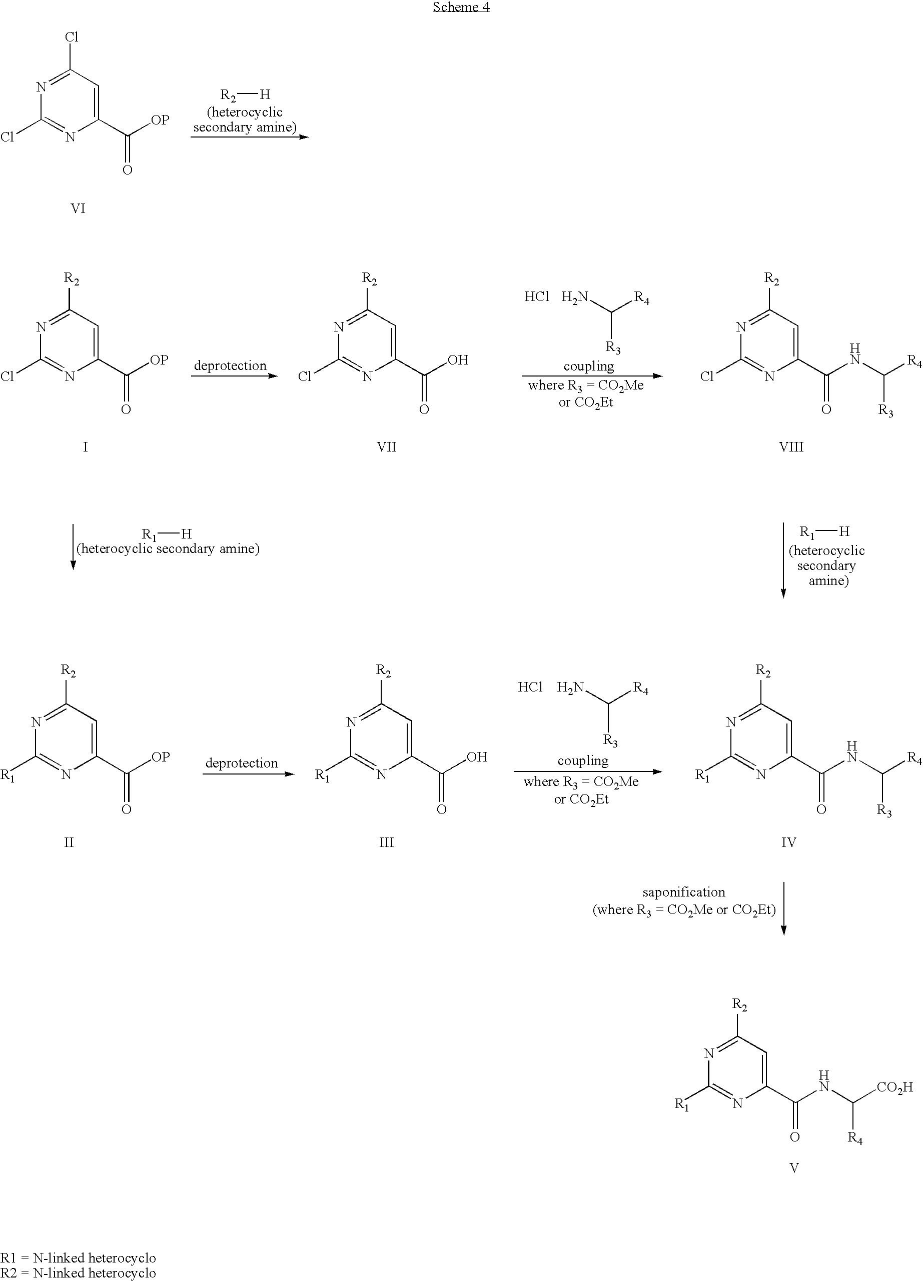

Pyrimidine (V) of formula 1, wherein Q₁ and Q₂ are N-linked heterocycles, can be prepared by the method outlined in Scheme 4. Reaction of dichloropyrimidine (VI) with a heterocyclic secondary amine, such as an optionally substituted piperidine or piperazine derivative, optionally in the presence of a suitable base, such as triethylamine, diisopropylethylamine, or the like, in an inert solvent, such as dichloromethane, chloroform, or tetrahydrofuran at a temperature ranging from −20° C. to 37° C. for from 1 to 24 hours, provides pyrimidine derivative (I). A second N-linked heterocycle can be introduced in a similar fashion to give compound (II). Removal of the ester protecting group of (II), for example by treatment with an acid, such as formic acid or trifluoroacetic acid, in the case of a t-butyl ester derivative, or by saponification with an alkali metal hydroxide, such as lithium hydroxide, sodium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, in the case of a methyl or ethyl ester, provides the corresponding acid derivative (III). The conversion of acid (III) to amide (IV) can be carried out by reaction of (III) with an amine nucleophile, such as an amino acid ester hydrochloride, and a suitable peptide coupling reagent, such as DCC, EDCI, PyBop, PyBrop, HATU, or the like, optionally in the presence of a suitable base, such as triethylamine, diisopropylethylamine, or the like, in an inert solvent, such as dichloromethane, chloroform, or tetrahydrofuran. The reaction is conducted at a temperature from −20° C. to 37° C. for from 2 to 48 hours. In the case where R₃ is an ester functionality, such as CO₂Me or CO₂Et, saponification with an alkali metal hydroxide, such as sodium hydroxide, lithium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, provides the corresponding acid derivative (V). It should be apparent to one skilled in the art that the order of steps in the reaction sequence can be altered (i.e., I→VII→VIII→IV→V) to obtain the same compounds of Formula 1 as shown in Scheme 4.

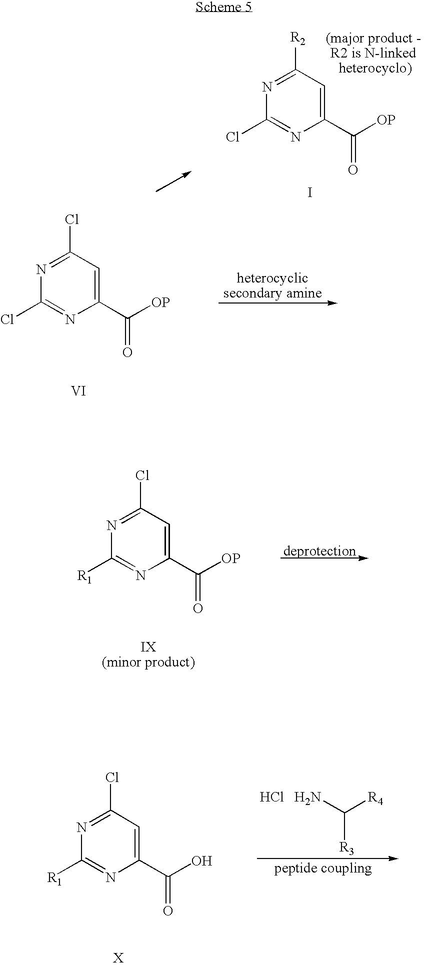

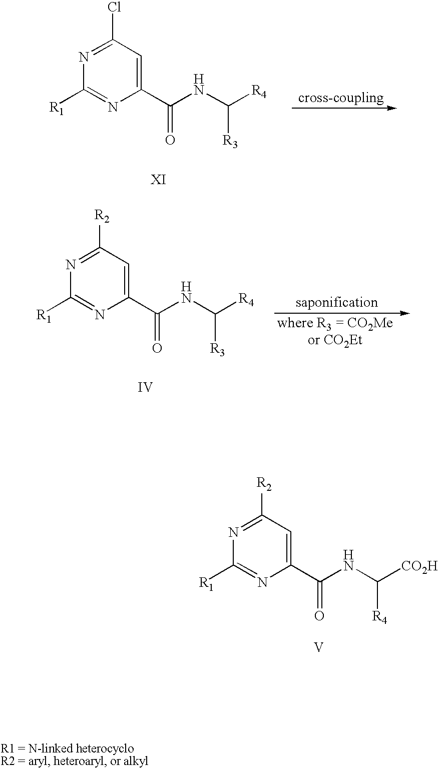

Pyrimidine (V) of Formula 1, wherein Q₁ is an N-linked heterocycle, Q₂ is a suitably substituted aryl, heteroaryl or alkyl group, and Q₃ is hydrogen, can be prepared by the method outlined in Scheme 5. Reaction of dichloropyrimidine (VI) with a heterocyclic secondary amine, such as an optionally substituted piperidine or piperazine derivative, optionally in the presence of a suitable base, such as triethylamine, diisopropylethylamine, or the like, in an inert solvent, such as dichloromethane, chloroform, or tetrahydrofuran at a temperature ranging from −20° C. to 37° C. for from 1 to 24 hours, can provide a mixture of two products. The major product (I) typically is that in which the heterocyclic secondary amine has added to the 4-position of (VI), as has been illustrated in Schemes 3 and 4. The minor product (IX) typically is that in which the heterocyclic secondary amine has added to the 2-position of (VI). Isolation of (IX) followed by removal of the ester protecting group, for example by treatment with an acid, such as formic acid or trifluoroacetic acid, in the case of a t-butyl ester derivative, or by saponification with an alkali metal hydroxide, such as lithium hydroxide, sodium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, in the case of a methyl or ethyl ester derivative, provides the corresponding acid derivative (X). The conversion of acid (X) to amide (XI) can be carried out by reaction of (X) with an amine nucleophile, such as an amino acid ester hydrochloride, and a suitable peptide coupling reagent, such as DCC, EDCI, PyBop, PyBrop, HATU, or the like, optionally in the presence of a suitable base, such as triethylamine, diisopropylethylamine, or the like, in an inert solvent, such as dichloromethane, chloroform, or tetrahydrofuran. The reaction is conducted at a temperature from −20° C. to 37° C. for from 2 to 48 hours. Cross-coupling reaction at the C-4 position of (XI) to give pyrimidine derivative (IV) can be achieved by one of the following methods:

1) a palladium-catalyzed Suzuki coupling reaction, with a suitably substituted aryl or heteroaryl boronic acid, catalyzed by, for example, tetrakis(triphenylphosphine)palladium(0) ((PPh₃)₄Pd), bis(dibenzylideneacetone)-palladium(0) (Pd(dba)₂), bis(tri-tert-butylphosphine)palladium(0) (Pd(P^{t}Bu₃)₂), or dichlorobis(triphenylphosphine)palladium(II) ((PPh₃)₂PdCl₂), using tris(o-furyl)-phosphine (TFP), triphenylphosphine (TPP) or 1,1′-bis(diphenylphosphino)-ferrocene (dppf) as ligand, in the presence of a base, such as, triethylamine, cesium fluoride, sodium carbonate or sodium tert-butoxide.

2) a Suzuki-Miyaura coupling reaction of an alkyl 9-BBN derivative catalyzed by, for example, PdCl₂(dppf), in the presence of a suitable base, such as cesium carbonate, sodium hydroxide, or the like.

Typically, these reactions are carried out in a suitable solvent, such as tetrahydrofuran, tetrahydrofuran/water mixture, methanol, dimethylformamide (DMF) or dimethoxyethane (DME), for from 1 to 48 hours at a temperature ranging from 0° C. to 80° C. Alternatively, these reactions can be conducted in a microwave in a sealed tube at an elevated temperature, ranging from 80° C. to 160° C. In the case where R₃ is an ester functionality, such as CO₂Me or CO₂Et, saponification with an alkali metal hydroxide, such as sodium hydroxide, lithium hydroxide or potassium hydroxide, in a suitable solvent, such as tetrahydrofuran, tetrahydrofuran/water mixture, ethanol, methanol, water, or an alcohol/water mixture, at a temperature ranging from 0° C. to 80° C. for from 1 to 48 hours, provides the corresponding acid derivative (V).

In certain preferred embodiments, compounds of the invention may be resolved to enantiomerically pure compositions or synthesized as enantiomerically pure compositions using any method known in art. By way of example, compounds of the invention may be resolved by direct crystallization of enantiomer mixtures, by diastereomer salt formation of enantiomers, by the formation and separation of diastereomers or by enzymatic resolution of a racemic mixture.

These and other reaction methodologies may be useful in preparing the compounds of the invention, as recognized by one of skill in the art. Various modifications to the above schemes and procedures will be apparent to one of skill in the art, and the invention is not limited specifically by the method of preparing the compounds of the invention.

C. Methods of the Invention

In another aspect of the invention, methods are provided for the inhibition of Type III protein section, and/or the treatment and prevention of bacterial infection, particularly Gram-negative bacterial infection using the compounds described herein.

In one embodiment, the invention is directed to methods for inhibiting Type III protein secretion comprising administering a secretion-inhibiting amount of at least one compound of the invention to a subject in need thereof.

In yet another embodiment, methods for treating or prevention bacterial infection, particularly Gram-Negative bacterial infection are provided comprising administering a therapeutically or prophylactically effective amount of at least one compound of the invention to a subject in need thereof.

According to the methods of the invention, the compound(s) may be administered to the subject via any drug delivery route known in the art. Specific exemplary administration routes include oral, ocular, rectal, buccal, topical, nasal, ophthalmic, subcutaneous, intramuscular, intravenous (bolus and infusion), intracerebral, transdermal, and pulmonary.

The terms “secretion-inhibiting amount”, “therapeutically effective amount”, and “prophylactically effective amount”, as used herein, refer to an amount of a compound of the invention sufficient to treat, ameliorate, or prevent the identified disease or condition, or to exhibit a detectable therapeutic, prophylactic, or inhibitory effect. The effect can be detected by, for example, the assays disclosed in the following examples. The precise effective amount for a subject will depend upon the subject's body weight, size, and health; the nature and extent of the condition; and the therapeutic or combination of therapeutics selected for administration. Therapeutically and prophylactically effective amounts for a given situation can be determined by routine experimentation that is within the skill and judgment of the clinician.

For any compound, the therapeutically or prophylactically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. The data obtained from cell culture assays and animal studies may be used in formulating a range of dosage for human use. The dosage contained in such compositions is preferably within a range of circulating concentrations that include an ED₅₀ with little or no toxicity. The dosage may vary within this range depending upon the dosage form employed, sensitivity of the patient, and the route of administration.

More specifically, the concentration-biological effect relationships observed with regard to the compound(s) of the present invention indicate an initial target plasma concentration ranging from approximately 5 μg/mL to approximately 100 μg/mL, preferably from approximately 10 μg/mL to approximately 100 μg/mL, more preferably from approximately 20 μg/mL to approximately 100 μg/mL. To achieve such plasma concentrations, the compounds of the invention may be administered at doses that vary from 0.1 μg to 100,000 mg, depending upon the route of administration. Guidance as to particular dosages and methods of delivery is provided in the literature and is generally available to practitioners in the art. In general the dose will be in the range of about 1 mg/day to about 10 g/day, or about 0.1 g to about 3g/day, or about 0.3 g to about 3 g/day, or about 0.5 g to about 2 g/day, in single, divided, or continuous doses for a patient weighing between about 40 to about 100 kg (which dose may be adjusted for patients above or below this weight range, particularly children under 40 kg).

The exact dosage will be determined by the practitioner, in light of factors related to the subject that requires treatment. Dosage and administration are adjusted to provide sufficient levels of the active agent(s) or to maintain the desired effect. Factors that may be taken into account include the severity of the disease state, general health of the subject, age, weight, and gender of the subject, diet, time and frequency of administration, drug, combination(s), reaction sensitivities, and tolerance/response to therapy. Long-acting pharmaceutical compositions may be administered every 3 to 4 days, every week, or once every two weeks depending on half-life and clearance rate of the particular formulation.

D. Metabolites of the Compounds of the Invention

Also falling within the scope of the present invention are the in vivo metabolic products of the compounds described herein. Such products may result for example from the oxidation, reduction, hydrolysis, amidation, esterification and the like of the administered compound, primarily due to enzymatic processes. Accordingly, the invention includes compounds produced by a process comprising contacting a compound of this invention with a mammalian tissue or a mammal for a period of time sufficient to yield a metabolic product thereof. Such products typically are identified by preparing a radio-labeled (e.g. C¹⁴ or H³) compound of the invention, administering it in a detectable dose (e.g., greater than about 0.5 mg/kg) to a mammal such as rat, mouse, guinea pig, monkey, or to man, allowing sufficient time for metabolism to occur (typically about 30 seconds to 30 hours), and isolating its conversion products from urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well known to those skilled in the art. The conversion products, so long as they are not otherwise found in vivo, are useful in diagnostic assays for therapeutic dosing of the compounds of the invention even if they possess no biological activity of their own.

E. Pharmaceutical Compositions of the Invention

While it is possible for the compounds of the present invention to be administered neat, it may be preferable to formulate the compounds as pharmaceutical compositions. As such, in yet another aspect of the invention, pharmaceutical compositions useful in the methods of the invention are provided. The pharmaceutical compositions of the invention may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH, and may range from a pH of about 3 to a pH of about 11, preferably about pH 3 to about pH 7, depending on the formulation and route of administration. In alternative embodiments, it may be preferred that the pH is adjusted to a range from about pH 5.0 to about pH 8.0.

More particularly, the pharmaceutical compositions of the invention comprise a therapeutically or prophylactically effective amount of at least one compound of the present invention, together with one or more pharmaceutically acceptable excipients. Optionally, the pharmaceutical compositions of the invention may comprise a combination of compounds of the present invention, or may include a second active ingredient useful in the treatment or prevention of bacterial infection (e.g., anti-bacterial or anti-microbial agents).

Formulations of the present invention, e.g., for parenteral or oral administration, are most typically solids, liquid solutions, emulsions or suspensions, while inhalable formulations for pulmonary administration are generally liquids or powders, with powder formulations being generally preferred. A preferred pharmaceutical composition of the invention may also be formulated as a lyophilized solid that is reconstituted with a physiologically compatible solvent prior to administration. Alternative pharmaceutical compositions of the invention may be formulated as syrups, creams, ointments, tablets, and the like.

The term “pharmaceutically acceptable excipient” refers to an excipient for administration of a pharmaceutical agent, such as the compounds of the present invention. The term refers to any pharmaceutical excipient that may be administered without undue toxicity. Pharmaceutically acceptable excipients are determined in part by the particular composition being administered, as well as by the particular method used to administer the composition. Accordingly, there exists a wide variety of suitable formulations of pharmaceutical compositions of the present invention (see, e.g., Remington's Pharmaceutical Sciences).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions of the invention may be formulated in any form suitable for the intended method of administration. When intended for oral use for example, tablets, troches, lozenges, aqueous or oil suspensions, non-aqueous solutions, dispersible powders or granules (including micronized particles or nanoparticles), emulsions, hard or soft capsules, syrups or elixirs may be prepared. Compositions intended for oral use may be prepared according to any method known to the art for the manufacture of pharmaceutical compositions, and such compositions may contain one or more agents including sweetening agents, flavoring agents, coloring agents and preserving agents, in order to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as croscarmellose sodium, cross-linked povidone, maize starch, or alginic acid; binding agents, such as povidone, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions of the invention may be formulated as suspensions comprising a compound of the present invention in admixture with at least one pharmaceutically acceptable excipient suitable for the manufacture of a suspension. In yet another embodiment, pharmaceutical compositions of the invention may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); and thickening agents, such as carbomer, beeswax, hard paraffin or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

Additionally, the pharmaceutical compositions of the invention may be in the form of a sterile injectable preparation, such as a sterile injectable aqueous emulsion or oleaginous suspension. This emulsion or suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,2-propane-diol. The sterile injectable preparation may also be prepared as a lyophilized powder. Among the acceptable vehicles and solvents that may be employed are water, Ringer's solution, and isotonic sodium chloride solution. In addition, sterile fixed oils may be employed as a solvent or suspending medium. For this purpose any bland fixed oil may be employed including synthetic mono- or diglycerides. In addition, fatty acids such as oleic acid may likewise be used in the preparation of injectables.

Generally, the compounds of the present invention useful in the methods of the present invention are substantially insoluble in water and are sparingly soluble in most pharmaceutically acceptable protic solvents and in vegetable oils. However, the compounds are generally soluble in medium chain fatty acids (e.g., caprylic and capric acids) or triglycerides and have high solubility in propylene glycol esters of medium chain fatty acids. Also contemplated in the invention are compounds which have been modified by substitutions or additions of chemical or biochemical moieties which make them more suitable for delivery (e.g., increase solubility, bioactivity, palatability, decrease adverse reactions, etc.), for example by esterification, glycosylation, PEGylation, etc.

In a preferred embodiment, the compounds of the present invention may be formulated for oral administration in a lipid-based formulation suitable for low solubility compounds. Lipid-based formulations can generally enhance the oral bioavailability of such compounds. As such; a preferred pharmaceutical composition of the invention comprises a therapeutically or prophylactically effective amount of a compound of the present invention, together with at least one pharmaceutically acceptable excipient selected from the group consisting of: medium chain fatty acids or propylene glycol esters thereof (e.g., propylene glycol esters of edible fatty acids such as caprylic and capric fatty acids) and pharmaceutically acceptable surfactants such as polyoxyl 40 hydrogenated castor oil.

In an alternative preferred embodiment, cyclodextrins may be added as aqueous solubility enhancers. Preferred cyclodextrins include hydroxypropyl, hydroxyethyl, glucosyl, maltosyl and maltotriosyl derivatives of α-, β-, and γ-cyclodextrin. A particularly preferred cyclodextrin solubility enhancer is hydroxypropyl-β-cyclodextrin (HPBC), which may be added to any of the above-described compositions to further improve the aqueous solubility characteristics of the compounds of the present invention. In one embodiment, the composition comprises 0.1% to 20% hydroxypropyl-β-cyclodextrin, more preferably 1% to 15% hydroxypropyl-β-cyclodextrin, and even more preferably from 2.5% to 10% hydroxypropyl-β-cyclodextrin. The amount of solubility enhancer employed will depend on the amount of the compound of the present invention in the composition.

F. Combination Therapy

It is also possible to combine any compound of the present invention with one or more other active ingredients useful in the treatment or prevention of bacterial infection, including compounds, in a unitary dosage form, or in separate dosage forms intended for simultaneous or sequential administration to a patient in need of treatment. When administered sequentially, the combination may be administered in two or more administrations. In an alternative embodiment, it is possible to administer one or more compounds of the present invention and one or more additional active ingredients by different routes.

The skilled artisan will recognize that a variety of active ingredients may be administered in combination with the compounds of the present invention that may act to augment or synergistically enhance the Type III protein secretion-inhibiting activity of the compounds of the invention.

According to the methods of the invention, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods of the invention may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

To assist in understanding the present invention, the following Examples are included. The experiments relating to this invention should not, of course, be construed as specifically limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof. The examples illustrate the preparation of certain compounds of the invention, and the testing of these compounds in vitro and/or in vivo. Those of skill in the art will understand that the techniques described in these examples represent techniques described by the inventors to function well in the practice of the invention, and as such constitute preferred modes for the practice thereof. However, it should be appreciated that those of skill in the art should in light of the present disclosure, appreciate that many changes can be made in the specific methods that are disclosed and still obtain a like or similar result without departing from the spirit and scope of the invention.

The following examples describe in detail the chemical synthesis of representative compounds of the present invention. The procedures are illustrations, and the invention should not be construed as being limited by the chemical reactions and conditions they express. No attempt has been made to optimize the yields obtained in these reactions, and it would be obvious to one skilled in the art that variations in reaction times, temperatures, solvents, and/or reagents could increase the yields.

### Example 1

### Preparation of Compounds of the Invention

Compounds of Formula I and Formula Ia may be prepared according to the schemes disclosed herein as follows.

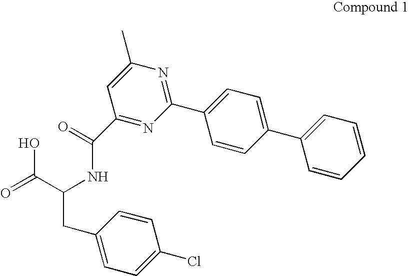

### 2-[(2-Biphenyl-4-yl-6-methyl-pyrimidine-4-carbonyl)amino]-3-(4-chlorophenyl)-propionic acid

### Step A: 2-Biphenyl-4-yl-6-methyl-pyrimidine-4-carboxylic acid methyl ester

To a mixture of 2-chloro-6-methyl-pyrimidine-4-carboxylic acid methyl ester (560 mg, 3.0 mmol), 4-biphenylboronic acid (653 mg, 3.3 mmol), tris(dibenzylideneacetone)dipalladium(0) (41 mg, 0.045 mmol) and tri-tert-butylphosphine (3 mol %) in THF (15 mL) at room temperature was added potassium fluoride (575 mg, 9.9 mmol) and the reaction mixture was heated at reflux temperature for 8 h. After cooling to room temperature, the mixture was filtered through a pad of Celite and the filtrate was concentrated in vacuo. Purification of the crude product by medium pressure liquid chromatography on silica gel (1:9 ethyl acetate/hexanes) gave 371 mg (41%) of the title compound.

### Step B: 2-Biphenyl-4-yl-6-methyl-pyrimidine-4-carboxylic acid

To a solution of 2-biphenyl-4-yl-6-methyl-pyrimidine-4-carboxylic acid methyl ester from Step A (371 mg, 1.2 mmol) in THF (3.0 mL) was added 1N lithium hydroxide (aq. 3.0 mL) and the resulting reaction mixture was stirred at room temperature for 3 h. After acidification with 4N hydrochloric acid to pH˜5, the THF was removed in vacuo and the aqueous layer was extracted with dichloromethane. The organic layer was separated, dried over sodium sulfate, filtered and the filtrate was concentrated in vacuo to give 314 mg (90%) of the title compound.

### Step C: 2-[(2-Biphenyl-4-yl -6-methyl-pyrimidine-4-carbonyl)amino]-3-(4-chloro-phenyl) propionic acid ethyl ester

To a mixture of 2-biphenyl-4-yl-6-methyl-pyrimidine-4-carboxylic acid from Step B (94 mg, 0.32 mmol), D,L-4-chlorophenylalanine ethyl ester hydrochloride (127 mg, 0.48 mmol), PyBrop (224 mg, 0.48 mmol) and dimethylaminopyridine (59 mg, 0.48 mmol) in dichloromethane (3.0 mL) at room temperature was added triethylamine (0.067 mL, 0.48 mmol) and the reaction mixture was stirred for 15 h. The reaction mixture was washed with water and the organic layer was concentrated in vacuo. Purification of the crude product by medium pressure liquid chromatography on silica gel (1:9 ethyl acetate/hexanes) gave 123 mg (77%) of the title compound.

### Step D: 2-[(2-Biphenyl-4-yl-6-methyl-pyrimidine-4-carbonyl)amino]-3-(4-chloro-phenyl) propionic acid

To a solution of 2-[(2-biphenyl-4-yl-6-methyl-pyrimidine-4-carbonyl)-amino]-3-(4-chlorophenyl)propionic acid ethyl ester from Step C (23 mg, 0.046 mmol) in THF (1.0 mL) was added 1N lithium hydroxide (aq. 1.0 mL) and the resulting reaction mixture was stirred at room temperature for 3 h. After acidification with 1N citric acid to pH˜5, the precipitate was collected by filtration to give 16 mg (74%) of the title compound. MS 472.1 [M+H]⁺.

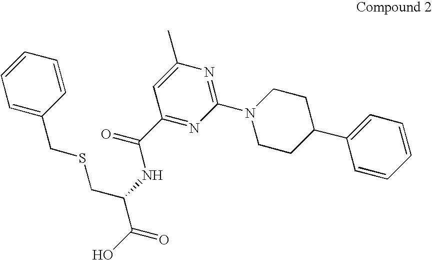

### (R)-3-Benzylsulfanyl-2-{[6-methyl-2-(4-phenylpiperidin-1-yl)pyrimidine-4-carbonyl]amino}propionic acid

### Step A: 6-Methyl-2-(4-phenylpiperidin-1-yl)pyrimidine-4-carboxylic acid methyl ester

To a solution of 2-chloro-6-methyl-pyrimidine-4-carboxylic acid methyl ester (186 mg, 1.0 mmol) in dichloromethane (5.0 mL) at 0° C. was added 4-phenylpiperidine (161 mg, 1.0 mmol) and the resulting reaction mixture was stirred at 0° C. for 30 min. and then at room temperature for 16 h. Saturated sodium bicarbonate (aq.) was added and the mixture was extracted with dichloromethane. The organic layer was separated, dried over sodium sulfate, filtered and the filtrate was concentrated in vacuo. Purification of the crude product by medium pressure liquid chromatography on silica gel (1:4 ethyl acetate/hexanes) gave 195 mg (63%) of the title compound.

### Step B: 6-Methyl-2-(4-phenylpiperidin-1-yl)pyrimidine-4-carboxylic acid

To a solution of 6-methyl-2-(4-phenylpiperidin-1-yl)pyrimidine-4-carboxylic acid methyl ester from Step A (195 mg, 0.63 mmol) in THF (5.0 mL)/MeOH (1.0 mL) was added 1N lithium hydroxide (aq. 2.0 mL) and the resulting reaction mixture was stirred at room temperature for 3 h. After acidification with 1N aq. citric acid to pH ˜5, the precipitate was collected by filtration to give 185 mg (99%) of the title compound.

### Step C: (R)-3-Benzylsulfanyl-2-{[6-methyl-2-(4-phenylpiperidin-1-yl)pyrimidine-4-carbonyl]amino}propionic acid methyl ester

To a mixture of 6-methyl-2-(4-phenylpiperidin-1-yl)pyrimidine-4-carboxylic acid from Step B (50 mg, 0.17 mmol), H-Cys(Bzl)-L-OMe hydrochloride (66 mg, 0.25 mmol), PyBrop (117 mg, 0.25 mmol) and dimethylaminopyridine (31 mg, 0.25 mmol) in dichloromethane (3.0 mL) at room temperature was added triethylamine (0.035 mL, 0.25 mmol) and the reaction mixture was stirred for 15 h. The reaction mixture was washed with water and the organic layer was concentrated in vacuo. Purification of the crude product by medium pressure liquid chromatography on silica gel (1:9 ethyl acetate/hexanes) gave 88 mg (100%) of the title compound.

### Step D: (R)-3-Benzylsulfanyl-2-{[6-methyl-2-(4-phenylpiperidin-1-yl)pyrimidine-4-carbonyl]amino}propionic acid

To a solution of (R)-3-benzylsulfanyl-2-{[6-methyl-2-(4-phenylpiperidin-1-yl)-pyrimidine-4-carbonyl]amino}propionic acid methyl ester from Step C (88 mg, 0.17 mmol) in THF (1.0 mL)/MeOH (1.0 mL) was added 1N lithium hydroxide (aq. 1.0 mL) and the resulting reaction mixture was stirred at room temperature for 16 h. After acidification with 1N citric acid (aq.) to pH ˜5, the precipitate was collected by filtration to give 80 mg (96%) of the title compound. MS 491.1 [M+H]⁺.

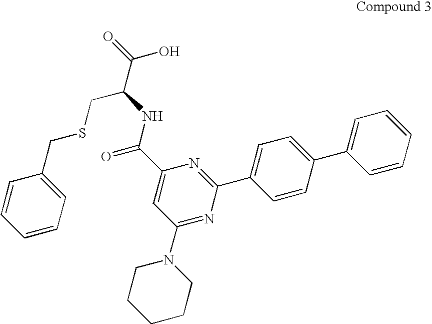

### (R)-3-Benzylsulfanyl-2-[(2-biphenyl-4-yl-6-piperidin-1-yl-pyrimidine-4-carbonyl)-amino]propionic acid

### Step A: 2-Chloro-6-piperidin-1-yl-pyrimidine-4-carboxylic acid methyl ester

To a solution of 2,6-dichloropyrimidine-4-carboxylic acid methyl ester (207 mg, 1.0 mmol) in dichloromethane (5.0 mL) at 0° C. was added piperidine (0.099 mL, 1.0 mmol) dropwise and the resulting reaction mixture was stirred at 0° C. for 30 min. and allowed to warm to room temperature. After 4 h the reaction mixture was washed with water and the organic layer was concentrated in vacuo. Purification of the crude product by medium pressure liquid chromatography on silica gel (1:9 ethyl acetate/hexanes) gave 103 mg (40%) of the title compound.

### Step B: 2-Biphenyl-4-yl-6-piperidin-1-yl-pyrimidine-4-carboxylic acid methyl ester

A mixture of 2-chloro-6-piperidin-1-yl-pyrimidine-4-carboxylic acid methyl ester from Step A (99 mg, 0.39 mmol), 4-biphenylboronic acid (154 mg, 0.78 mmol), tris(dibenzylideneacetone)dipalladium(0) (7 mg, 1 mol %), tri-tert-butylphosphine (2 mol %) and potassium fluoride (45 mg, 0.78 mmol) in THF (3.0 mL) was heated in a microwave oven at 120° C. for 20 min. The reaction mixture was then filtered through a pad of Celite and the filtrate was concentrated in vacuo. Purification of the crude product by medium pressure liquid chromatography on silica gel (1:9 ethyl acetate/hexanes) gave 75 mg (52%) of the title compound.

### Step C: 2-Biphenyl-4-yl-6-piperidin-1-yl-pyrimidine-4-carboxylic acid

To a solution of 2-biphenyl-4-yl-6-piperidin-1-yl-pyrimidine-4-carboxylic acid methyl ester from Step B (70 mg, 0.18 mmol) in THF (1.0 mL)/MeOH (1.0 mL) was added 1N lithium hydroxide (aq. 1.0 mL) and the resulting reaction mixture was stirred at room temperature for 4 h. After acidification with 1N hydrochloric acid (aq.) to pH˜5, the white precipitate was collected by filtration to give 30 mg (46%) of the title compound.

### Step D: (R)-3-Benzylsulfanyl-2-[(2-biphenyl-4-yl-6-piperidin-1-yl-pyrimidine-4-carbonyl)amino]propionic acid methyl ester

To a mixture of 2-biphenyl-4-yl-6-piperidin-1-yl-pyrimidine-4-carboxylic acid from Step C (26 mg, 0.072 mmol), H-Cys(Bzl)-L-OMe hydrochloride (29 mg, 0.11 mmol), PyBrop (51 mg, 0.11 mmol) and dimethylaminopyridine (13 mg, 0.11 mmol) in dichloromethane (2.0 mL) at room temperature was added triethylamine (0.015 mL, 0.11 mmol) and the reaction mixture was stirred for 16 h. The reaction mixture was washed with water and the organic layer was concentrated in vacuo. Purification of the crude product by medium pressure liquid chromatography on silica gel (1:9 ethyl acetate/hexanes) gave 30 mg (74%) of the title compound.

### Step E: (R)-3-Benzylsulfanyl-2-[(2-biphenyl-4-yl-6-piperidin-1-yl-pyrimidine-4-carbonyl)amino]propionic acid

To a solution of (R)-3-benzylsulfanyl-2-[(2-biphenyl-4-yl-6-piperidin-1-yl-pyrimidine-4-carbonyl)amino]propionic acid methyl ester from Step D (26 mg, 0.046 mmol) in THF (1.0 mL)/MeOH (1.0 mL) was added 1N lithium hydroxide (aq. 0.5 mL) and the resulting reaction mixture was stirred at room temperature for 3 h. After acidification with 1N hydrochloric acid (aq.) to pH˜5, the precipitate was collected by filtration to give 24 mg (94%) of the title compound. MS 553.0 [M+H]⁺.

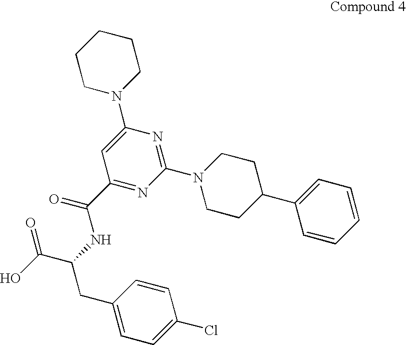

### (R)-3-(4-Chlorophenyl)-2-{[2-(4-phenylpiperidin-1-yl)-6-piperidin-1-yl-pyrimidine-4-carbonyl]amino}propionic acid

### Step A: 2-Chloro-6-piperidin-1-yl-pyrimidine-4-carboxylic acid methyl ester

To a solution of 2,6-dichloropyrimidine-4-carboxylic acid methyl ester (1.0 g, 5.0 mmol) in dichloromethane (20.0 mL) at 0° C. was added triethylamine (0.70 mL, 5.0 mmol) followed by piperidine (0.49 mL, 5.0 mmol) and the resulting reaction mixture was stirred at 0° C. for 30 min. and allowed to warm to room temperature. After 3 h the reaction mixture was washed with water and the organic layer was concentrated in vacuo. Purification of the crude product by medium pressure liquid chromatography on silica gel (1:4 ethyl acetate/hexanes) gave 1.1 g (86%) of the title compound.

### Step B: 2-(4-Phenylpiperidin-1-yl)-6-1yl-piperidin-1-yl-pyrimidine-4-carboxylic acid methyl ester

To a solution of 2-chloro-6-piperidin-1-yl-pyrimidine-4-carboxylic acid methyl ester from Step A (256 mg, 1.0 mmol) in dichloromethane (10.0 mL) at 0° C. was added triethylamine (0.14 mL, 1.0 mmol) followed by 4-phenylpiperidine (322, 2.0 mmol) and the resulting reaction mixture was stirred at 0° C. for 30 min. and allowed to warm to room temperature. After 3 h the reaction mixture was washed with water and the organic layer was concentrated in vacuo. Purification of the crude product by medium pressure liquid chromatography on silica gel (1:4 ethyl acetate/hexanes) gave 240 mg (63%) of the title compound.

### Step C: 2-(4-Phenylpiperidin-1yl)-6-piperidin-1-yl-pyrimidine-4-carboxylic acid

To a solution of 2-(4-phenylpiperidin-1-yl)-6-piperidin-1-yl-pyrimidine-4-carboxylic acid methyl ester from Step B (240 mg, 0.64 mmol) in THF (1.0 mL)/MeOH (1.0 mL) was added 1N lithium hydroxide (aq. 1.0 mL) and the resulting reaction mixture was stirred at room temperature for 4 h. After acidification with 1N citric acid (aq.) to pH˜5, the white precipitate was collected by filtration to give 230 mg (98%) of the title compound.

### Step D: (R)-3-(4-Chlorophenyl)-2-{[2-(4-phenylpiperidin-1-yl)-6-piperidin-1-yl-pyrimidine-4-carbonyl]amino}propionic acid methyl ester

To a mixture of 2-(4-phenylpiperidin-1-yl)-6-piperidin-1-yl-pyrimidine-4-carboxylic acid from Step C (50 mg, 0.14 mmol), D-4-chlorophenylalanine methyl ester hydrochloride (50 mg, 0.20 mmol), PyBrop (93 mg, 0.20 mmol) and dimethylaminopyridine (24 mg, 0.20 mmol) in dichloromethane (2.0 mL) at room temperature was added triethylamine (0.028 mL, 0.20 mmol) and the reaction mixture was stirred for 8 h. The reaction mixture was washed with water and the organic layer was concentrated in vacuo. Purification of the crude product by medium pressure liquid chromatography on silica gel (1:4 ethyl acetate/hexanes) gave 51 mg (65%) of the title compound.

### Step E: (R)-3-(4-Chlorophenyl)-2-{[2-(4-phenylpiperidin-1-yl)-6-piperidin-1-yl-pyrimidine-4-carbonyl]amino}propionic acid

To a solution of (R)-3-(4-chlorophenyl)-2-{[2-(4-phenylpiperidin-1-yl)-6-piperidin-1-yl-pyrimidine-4-carbonyl]amino}propionic acid methyl ester from Step D (51 mg, 0.09 mmol) in dichloromethane (0.5 mL)/MeOH (1.0 mL) was added 1N sodium hydroxide (aq. 0.5 mL) and the resulting reaction mixture was stirred at room temperature for 3 h. After acidification with 1N citric acid (aq.) to pH˜5, the precipitate was collected by filtration to give 34 mg (69%) of the title compound. MS 548.3 [M+H]⁺.

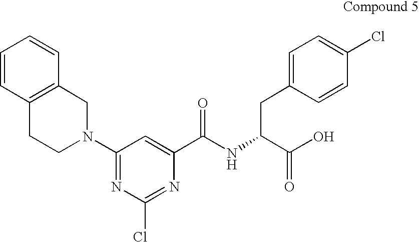

### (R)-2-{[2-Chloro-6-(3,4-dihydro-1H-isoquinolin-2-yl)pyrimidine-4-carbonyl]amino}-3-(4-chlorophenyl)propionic acid

### Step A: 2-Chloro-6-(3,4-dihydro-1H-isoquinolin-2-yl)pyrimidine-4-carboxylic acid methyl ester

To a solution of 2,6-dichloropyrimidine-4-carboxylic acid methyl ester (414 mg, 2.0 mmol) in dichloromethane (10.0 mL) at 0° C. was added 1,2,3,4-tetrahydro isoquinoline (0.25 mL, 2.0 mmol) and the resulting reaction mixture was stirred at 0° C. for 30 min. and allowed to warm to room temperature. After 3 h the reaction mixture was washed with water and the organic layer was concentrated in vacuo. Purification of the crude product by medium pressure liquid chromatography on silica gel (1:4 ethyl acetate/hexanes) gave 380 mg (63%) of the title compound.

### Step B: 2-Chloro-6-(3,4-dihydro-1H-isoquinolin-2-yl)pyrimidine-4-carboxylic acid

To a solution of 2-chloro-6-(3,4-dihydro-1H-isoquinolin-2-yl)pyrimidine-4-carboxylic acid methyl ester from Step A (278 mg, 0.92 mmol) in THF (2.0 mL)/MeOH (1.0 mL)/dichloromethane (1.0 mL) was added 1N sodium hydroxide (aq. 3.0 mL) and the resulting reaction mixture was stirred at room temperature for 24 h. After solvents were removed in vacuo, the reaction mixture was acidified with 1N citric acid to pH 5, and the aqueous layer was extracted with dichloromethane. The organic layer was separated, dried over sodium sulfate, filtered and the filtrate was concentrated in vacuo to give 265 mg (99%) of the title compound.

### Step C: (R)-2-{[2-Chloro-6-(3,4-dihydro-1H-isoquinolin-2-yl)pyrimidine-4-carbonyl]amino}-3-(4-chlorophenyl)propionic acid methyl ester

To a mixture of 2-chloro-6-(3,4-dihydro-1H-isoquinolin-2-yl)pyrimidine-4-carboxylic acid from Step B (265 mg, 0.92 mmol), D-4-chlorophenylalanine methyl ester hydrochloride (343 mg, 1.37 mmol), PyBrop (639 mg, 137 mmol) and dimethylaminopyridine (167 mg, 1.37 mmol) in dichloromethane (15.0 mL) at room temperature was added triethylamine (0.19 mL, 1.37 mmol) and the reaction mixture was stirred for 15 h. The reaction mixture was washed with water and the organic layer was concentrated in vacuo. Purification of the crude product by medium pressure liquid chromatography on silica gel (1:4 ethyl acetate/hexanes) gave 301 mg (68%) of the title compound.

### Step D: (R)-2-{[2-Chloro-6-(3,4-dihydro-1H-isoquinolin-2-yl)pyrimidine-4-carbonyl]-amino}-3-(4-chlorophenyl)propionic acid

To a solution of (R)-2-{[2-chloro-6-(3,4-dihydro-1H-isoquinolin-2-yl)pyrimidine-4-carbonyl]amino}-3-(4-chlorophenyl)propionic acid methyl ester from Step C (49 mg, 0.1 mmol) in methanol (1.0 mL) was added 1N lithium hydroxide (aq. 1.0 mL) and the resulting reaction mixture was stirred at room temperature for 2 h. After acidification with 1N citric acid (aq.) to pH ˜5, the white precipitate was collected by filtration to give 46 mg (98%) of the title compound. MS 471.2 [M+H]⁺.

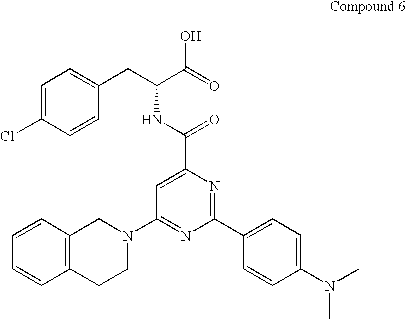

### (R)-3-(4-Chlorophenyl)-2-{[6-(3,4-dihydro-1H-isoquinolin-2-yl)-2-(4-dimethylaminophenyl)pyrimidine-4-carbonyl]amino}propionic acid

### Step A: (R)-3-(4-Chlorophenyl)-2-{[6-(3,4-dihydro-1H-isoquinolin-2-yl)-2-(4-dimethyl aminophenyl)pyrimidine-4-carbonyl]amino}propionic acid methyl ester

A mixture of (R)-2-{[2-chloro-6-(3,4-dihydro-1H-isoquinolin-2-yl)pyrimidine-4-carbonyl]amino}-3-(4-chlorophenyl)propionic acid methyl ester from Step C of Compound 5 (49 mg, 0.1 mmol), 4-dimethylaminophenyl boronic acid (83 mg, 0.5 mmol), dichlorobis(triphenylphosphine)palladium(II) (7 mg, 10 mol %) and triethylamine (0.07 mL, 0.5 mmol) in methanol (2.5 mL) was heated in a microwave oven at 150° C. for 20 min. The reaction mixture was then filtered through a pad of Celite and the filtrate was concentrated in vacuo. Purification of the crude product by medium pressure liquid chromatography on silica gel (1:4 ethyl acetate/hexanes) gave 40 mg (70%) of the title compound.

### Step B: (R)-3-(4-Chlorophenyl)-2-{[6-(3,4-dihydro-1H-isoquinolin-2-yl)-2-(4-dimethylaminophenyl)pyrimidine-4-carbonyl]amino}propionic acid

To a solution of (R)-3-(4-chlorophenyl)-2-{[6-(3,4-dihydro-1H-isoquinolin-2-yl)-2-(4-dimethylaminophenyl)pyrimidine-4-carbonyl]amino}propionic acid methyl ester from Step A (35 mg, 0.06 mmol) in THF (1.0 mL) was added 1N lithium hydroxide (aq. 1.0 mL) and the resulting reaction mixture was stirred at room temperature for 2 h. After acidification with 1N citric acid (aq.) to pH˜5, the precipitate was collected by filtration to give 18 mg (54%) of the title compound. MS 556.2 [M+H]⁺.

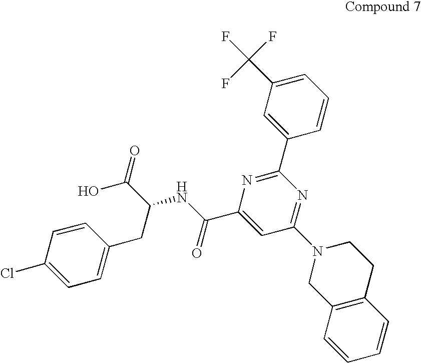

### (R)-3-(4-Chlorophenyl)-2-{[6-(3,4-dihydro-1H-isoquinolin-2-yl)-2-(3-trifluoromethylphenyl)pyrimidine-4-carbonyl]amino}propionic acid

The title compound was prepared by a procedure analogous to that of Compound 6 by substituting 3-trifluoromethylphenyl boronic acid for 4-dimethylaminophenyl-boronic acid of Step A of Compound 6. MS 581.1 (M+H)⁺.

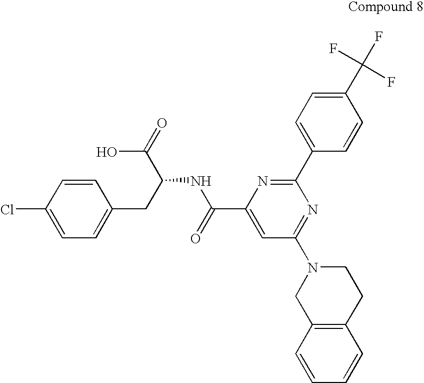

### (R)-3-(4-Chlorophenyl)-2-{[6-(3,4-dihydro-1H-isoquinolin-2-yl)-2-(4-trifluoromethylphenyl)pyrimidine-4-carbonyl]amino}propionic acid

The title compound was prepared by a procedure analogous to that of Compound 6 by substituting 4-trifluoromethylphenyl boronic acid for 4-dimethylamino-phenylboronic acid of Step A of Compound 6. MS 581.1 (M+H)⁺.

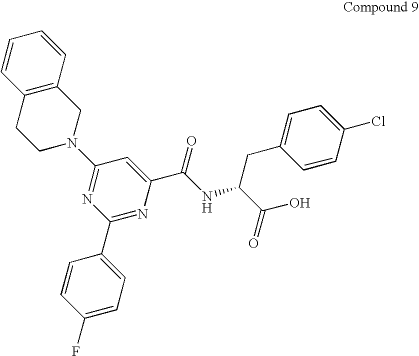

### (R)-3-(4-Chlorophenyl)-2-{[6-(3,4-dihydro-1H-isoquinolin-2-yl)-2-(4-fluoro-phenyl)Pyrimidine-4-carbonyl]amino}propionic acid

The title compound was prepared by a procedure analogous to that of Compound 6 by substituting 4-fluorophenylboronic acid for 4-dimethylaminophenyl boronic acid of Step A of Compound 6. MS 531.0 (M+H)⁺.

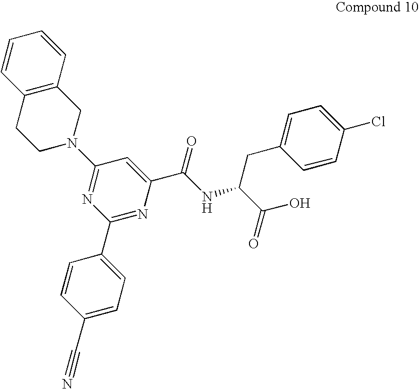

### (R)-3-(4-Chlorophenyl)-2-{[2-(4-cyanophenyl)-6-(3,4-dihydro-1H-isoquinolin-2-yl)pyrimidine-4-carbonyl]amino}propionic acid

The title compound was prepared by a procedure analogous to that of Compound 6 by substituting 4-cyano phenylboronic acid for 4-dimethylaminophenyl boronic acid of Step A of Compound 6. MS 538.0 (M+H)⁺.

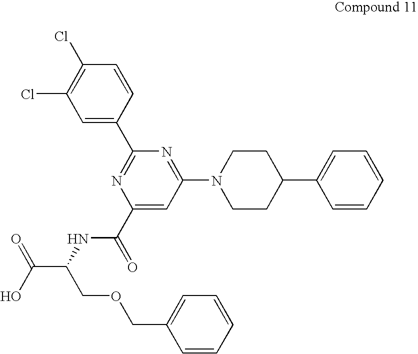

### (R)-3-Benzyloxy-2-{[2-(3,4-dichlorophenyl)-6-(4-phenylpiperidin-1-yl)-pyrimidine-4-carbonyl]amino}propionic acid

The title compound was prepared by procedures analogous to those of Compound 5 and Compound 6 by substituting 4-phenylpiperidine for 1,2,3,4,-tetrahydro-isoquinoline of Step A of Compound 5, by substituting O-benzyl-D-serine methyl ester hydrochloride for D-4-chlorophenylalanine of Step C of Compound 5, and by substituting 3,4-dichlorophenyl boronic acid for 4-dimethylaminophenyl boronic acid of Step A of Compound 6. MS 605.0 (M+H)⁺.

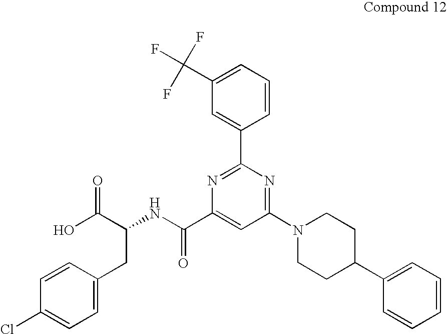

### (R)-3-(4-Chlorophenyl)-2-{[6-(4-phenylpiperidin-1-yl)-2-(3-trifluoromethyl-phenyl)pyrimidine-4-carbonyl]amino}propionic acid

The title compound was prepared by procedures analogous to those of Compound 5 and Compound 6 by substituting 4-phenylpiperidine for 1,2,3 4,-tetrahydro-isoquinoline of Step A of Compound 5; and by substituting 3-trifluoromethylphenyl boronic acid for 4-dimethylaminophenyl boronic acid of Step A of Compound 6. MS 609.0 (M+H)⁺.

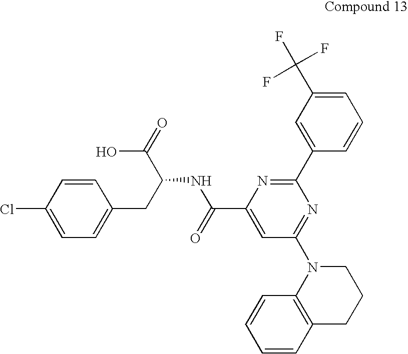

### (R)-3-(4-Chlorophenyl)-2-{[6-(3,4-dihydro-2H-quinolin-1-yl)-2-(3-trifluoromethyl-phenyl)pyrimidine-4-carbonyl]amino}propionic acid

The title compound was prepared by procedures analogous to those of Compound 5 and Compound 6 by substituting 1,2,3,4,-tetrahydro quinoline-for 1,2,3,4,-tetrahydro isoquinoline of Step A of Compound 5, and by substituting 3-trifluoromethylphenyl boronic acid for 4-dimethylaminophenyl boronic acid of Step A of Compound 6. MS 581.1 (M+H)⁺.

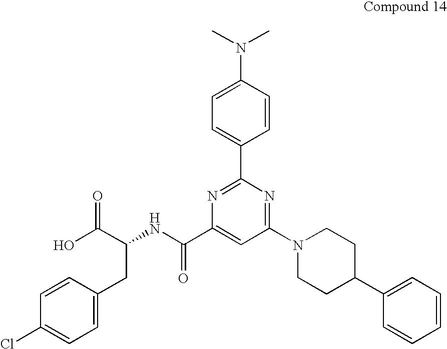

### (R)-3-(4-Chlorophenyl)-2-{[2-(4-dimethylaminophenyl)-6-(4-phenylpiperidin-1-yl)pyrimidine-4-carbonyl]amino}propionic acid

The title compound was prepared by procedures analogous to those of Compound 5 and Compound 6 by substituting 4-phenylpiperidine for 1,2,3,4,-tetrahydro-isoquinoline of Step A of Compound 5. MS 584.1 (M+H)⁺.

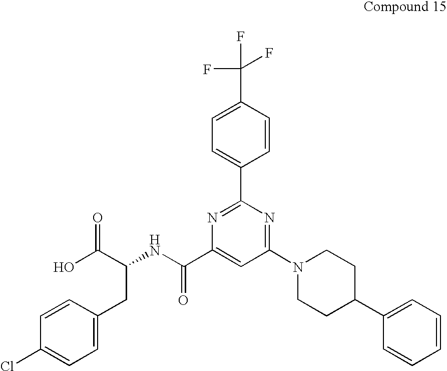

### (R)-3-(4-Chlorophenyl)-2-{[6-(4-phenylpiperidin-1-yl)-2-(4-trifluoromethylphenyl)-pyrimidine-4-carbonyl]amino}propionic acid

The title compound was prepared by procedures analogous to those of Compound 5 and Compound 6 by substituting 4-phenylpiperidine for 1,2,3,4,-tetrahydro-isoquinoline of Step A of Compound 5, and by substituting 4-trifluoromethylphenyl boronic acid for 4-dimethylaminophenyl boronic acid of Step A of Compound 6. MS 609.0 (M+H)⁺.

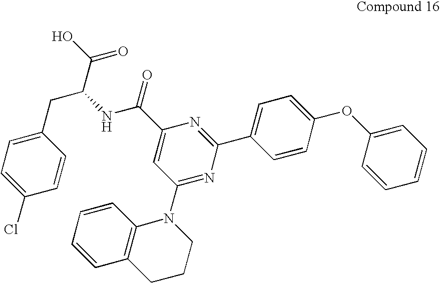

### (R)-3-(4-Chlorophenyl)-2-{[6-(3,4-dihydro-2H-quinolin-1-yl)-2-(4-phenoxy-phenyl)pyrimidine-4-carbonyl]amino}propionic acid

The title compound was prepared by procedures analogous to those of Compound 5 and Compound 6 by substituting 1,2,3,4,-tetrahydro quinoline for 1,2,3,4,-tetrahydro isoquinoline of Step A of Compound 5, and by substituting 4-phenoxyphenylboronic acid for 4-dimethylaminophenyl boronic acid of Step A of Compound 6. MS 605.1 (M+H)⁺.

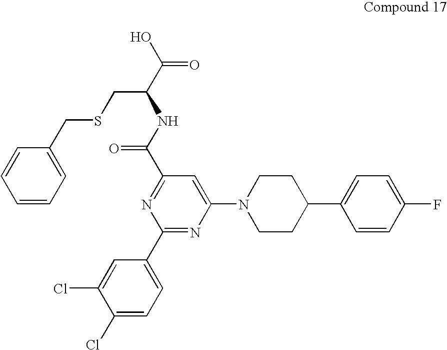

### (R)-3-Benzylsulfanyl-2-({2-(3,4-dichlorophenyl)-6-[4-(4-fluorophenyl)piperidin-1-yl]pyrimidine-4-carbonyl}amino)propionic acid

The title compound was prepared by procedures analogous to those of Compound 5 and Compound 6 by substituting 4-(4′-fluorophenyl)piperidine for 1,2,3,4,-tetrahydro isoquinoline of Step A of Compound 5, by substituting H-Cys(Bzl)-L-OMe hydrochloride for D-4-chlorophenylalanine of Step C of Compound 5, and by substituting 3,4-dichlorophenyl boronic acid for 4-dimethylaminophenyl boronic acid of Step A of Compound 6. MS 639.2 (M+H)+. 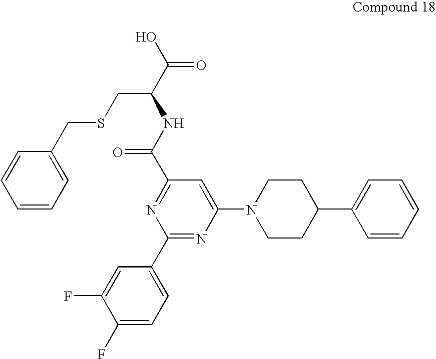
(R)-3-Benzylsulfanyl-2-{[2-(3,4-difluorophenyl)-6-(4-phenylpiperidin-1-yl)-pyrimidine-4-carbonyl]amino}propionic acid

The title compound was prepared by procedures analogous to those of Compound 5 and Compound 6 by substituting 4-phenylpiperidine for 1,2,3,4,-tetrahydro-isoquinoline of Step A of Compound 5, by substituting H-Cys(Bzl)-L-OMe hydrochloride for D-4-chlorophenylalanine of Step C of Compound 5, and by substituting 3,4-difluorophenyl boronic acid for 4-dimethylaminophenyl boronic acid of Step A of Compound 6. MS 589.0 (M+H)⁺.

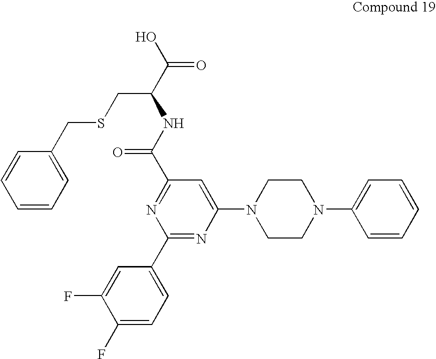

### (R)-3-Benzylsulfanyl-2-{[2-(3,4-difluorophenyl)-6-(4-phenyl-piperazin-1-yl)-pyrimidine-4-carbonyl]amino}propionic acid

The title compound was prepared by procedures analogous to those of Compound 5 and Compound 6 by substituting 4-phenyl-piperazine for 1,2,3,4,-tetrahydro-isoquinoline of Step A of Compound 5, by substituting H-Cys(Bzl)-L-OMe hydrochloride for D-4-chlorophenylalanine of Step C of Compound 5, and by substituting 3,4-difluorophenyl boronic acid for 4-dimethylaminophenyl boronic acid of Step A of Compound 6. MS 590.2 (M+H)⁺.

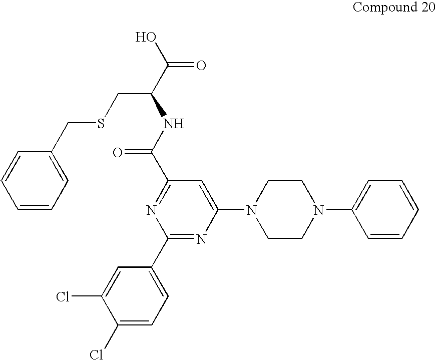

### (R)-3-Benzylsulfanyl-2-{[2-(3,4-dichlorophenyl)-6-(4-phenyl-piperazin-1-yl)-pyrimidine-4-carbonyl]amino}propionic acid

The title compound was prepared by procedures analogous to those of Compound 5 and Compound 6 by substituting 4-phenyl-piperazine for 1,2,3,4,-tetrahydro-isoquinoline of Step A of Compound 5, by substituting H-Cys(Bzl)-L-OMe hydrochloride for D-4-chlorophenylalanine of Step C of Compound 5, and by substituting 3,4-dichlorophenyl boronic acid for 4-dimethylaminophenyl boronic acid of Step A of Compound 6. MS 622.0 (M+H)⁺.

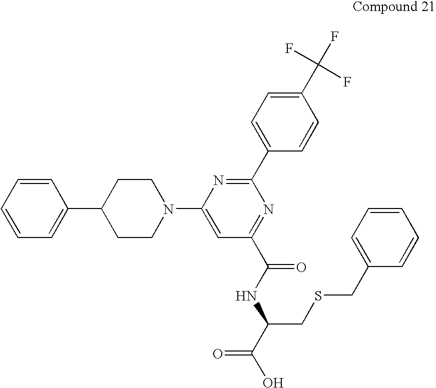

### (R)-3-Benzylsulfanyl-2-{[6-(4-phenylpiperidin-1-yl)-2-(4-trifluoromethylphenyl)-pyrimidine-4-carbonyl]amino}propionic acid

The title compound was prepared by procedures analogous to those of Compound 5 and Compound 6 by substituting 4-phenylpiperidine for 1,2,3,4,-tetrahydro-isoquinoline of Step A of Compound 5, by substituting H-Cys(Bzl)-L-OMe hydrochloride for D-4-chlorophenylalanine of Step C of Compound 5, and by substituting 4-trifluoromethylphenyl boronic acid for 4-dimethylaminophenyl-boronic acid of Step A of Compound 6. MS 621.2 (M+H)⁺.

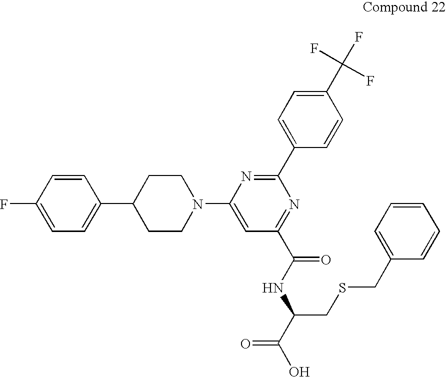

### (R)-3-Benzylsulfanyl-2-{[6-[4-(4-fluorophenyl)piperidin-1-yl]-2-(4-trifluoromethylphenyl)pyrimidine-4-carbonyl]amino}propionic acid

The title compound was prepared by procedures analogous to those of Compound 5 and Compound 6 by substituting 4-(4′-fluorophenyl)piperidine for 1,2,3,4,-tetrahydro isoquinoline of Step A of Compound 5, by substituting H-Cys(Bzl)-L-OMe hydrochloride for D-4-chlorophenylalanine of Step C of Compound 5, and by substituting 4-trifluoromethylphenyl boronic acid for 4-dimethylaminophenyl-boronic acid of Step A of Compound 6. MS 639.2 (M+H)⁺.

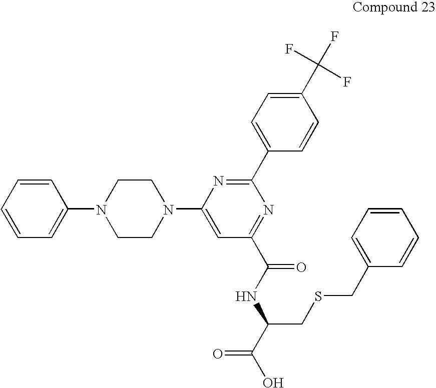

### (R)-3-Benzylsulfanyl-2-{[6-(4-phenyl-piperazin-1-yl)-2-(4-trifluoromethylphenyl)-pyrimidine-4-carbonyl]amino}propionic acid

The title compound was prepared by procedures analogous to those of Compound 5 and Compound 6 by substituting 4-phenyl-piperazine for 1,2,3,4,-tetrahydro-isoquinoline of Step A of Compound 5, by substituting H-Cys(Bzl)-L-OMe hydrochloride for D-4-chlorophenylalanine of Step C of Compound 5, and by substituting 4-trifluoromethylphenyl boronic acid for 4-dimethylaminophenyl-boronic acid of Step A of Compound 6. MS 622.2 (M+H)⁺.

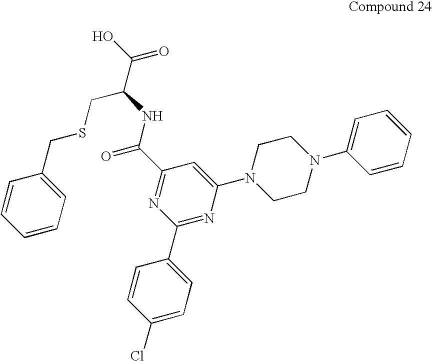

### (R)-3-Benzylsulfanyl-2-{[2-(4-chlorophenyl)-6-(4-phenyl-piperazin-1-yl)-pyrimidine-4-carbonyl]amino}propionic acid

The title compound was prepared by procedures analogous to those of Compound 5 and Compound 6 by substituting 4-phenyl-piperazine for 1,2;3,4,-tetrahydro-isoquinoline of Step A of Compound 5, by substituting H-Cys(Bzl)-L-OMe hydrochloride for D-4-chlorophenylalanine of Step C of Compound 5, and by substituting 4-chlorophenyl boronic acid for 4-dimethylaminophenyl boronic acid of Step A of Compound 6. MS 588.2 (M+H)⁺.

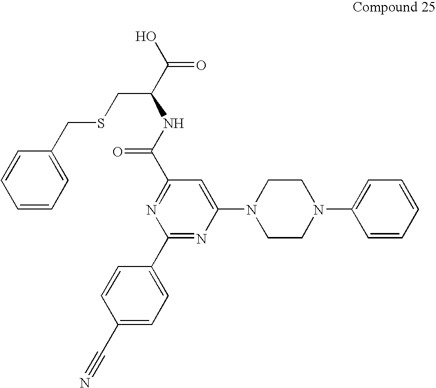

### (R)-3-Benzylsulfanyl-2-{[2-(4-cyanophenyl)-6-(4-phenyl-piperazin-1-yl)-pyrimidine-4-carbonyl]amino}propionic acid

The title compound was prepared by procedures analogous to those of Compound 5 and Compound 6 by substituting 4-phenyl-piperazine for 1,2,3,4,-tetrahydro-isoquinoline of Step A of Compound 5, by substituting H-Cys(Bzl)-L-OMe hydrochloride for D-4-chlorophenylalanine of Step C of Compound 5, and by substituting 4-cyanophenyl boronic acid for 4-dimethylaminophenyl boronic acid of Step A of Compound 6. MS 579.2 (M+H)⁺.

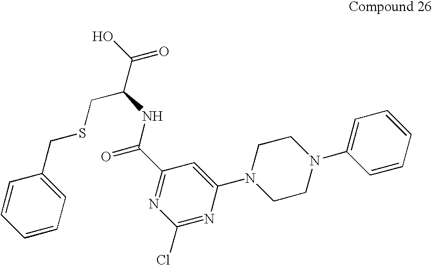

### (R)-3-Benzylsulfanyl-2-{[2-chloro-6-(4-phenyl-piperazin-1-yl)pyrimidine-4-carbonyl]amino}propionic acid

The title compound was prepared by a procedure analogous to that of Compound 5 by substituting 4-phenyl-piperazine for 1,2,3,4,-tetrahydro isoquinoline of Step A of Compound 5, and by substituting H-Cys(Bzl)-L-OMe hydrochloride for D-4-chlorophenylalanine of Step C of Compound 5. MS 512.1 (M+H)⁺.

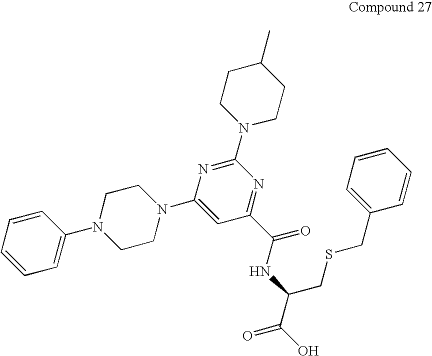

### (R)-3-Benzylsulfanyl-2-{[2-(4-methylpiperidin-1-yl)-6-(4-phenyl-piperazin-1-yl)-pyrimidine-4-carbonyl]amino}propionic acid

The title compound was prepared by a procedure analogous to that of Compound 4 by substituting 4-phenyl-piperazine for piperidine of Step A of Compound 4 by substituting 4-methyl-piperidine for 4-phenylpiperidine of Step B of Compound 4 and by substituting H-Cys(Bzl)-L-OMe hydrochloride for D-4-chlorophenylalanine of Step D of Compound 4. MS 575.2 (M+H)⁺.

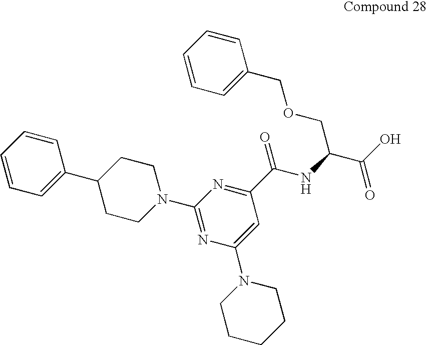

### (S)-3-Benzyloxy-2-{[2-(4-phenylpiperidin-1-yl)-6-piperidin-1-yl-pyrimidine-4-carbonyl]amino}propionic acid

The title compound was prepared by a procedure analogous to that of Compound 4 by substituting O-benzyl-L-serine methyl ester hydrochloride for D-4-chlorophenylalanine of Step D of Compound 4. MS 544.2 (M+H)⁺.

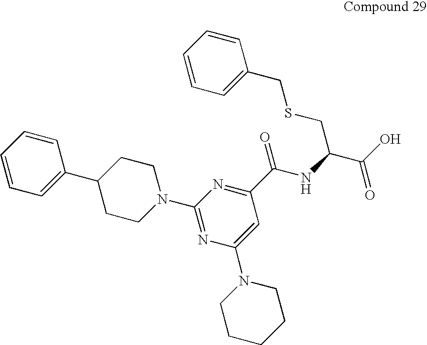

### (R)-3-Benzylsulfanyl-2-{[2-(4-phenylpiperidin-1-yl)-6-piperidin-1-yl-pyrimidine-4-carbonyl]amino}propionic acid

The title compound was prepared by a procedure analogous to that of Compound 4 by substituting H-Cys(Bzl)-L-OMe hydrochloride for D-4-chlorophenylalanine of Step D of Compound 4. MS 560.2 (M+H)⁺.

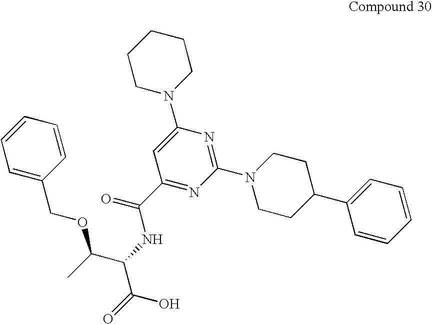

### (2S,3R)-3-Benzyloxy-2-{[2-(4-phenylpiperidin-1-yl)-6-piperidin-1-yl-pyrimidine-4-carbonyl]amino}butyric acid

The title compound was prepared by a procedure analogous to that of Compound 4 by substituting O-benzyl-L-threonine methyl ester hydrochloride for D-4-chlorophenylalanine of Step D of Compound 4. MS 558.3 (M+H)⁺.

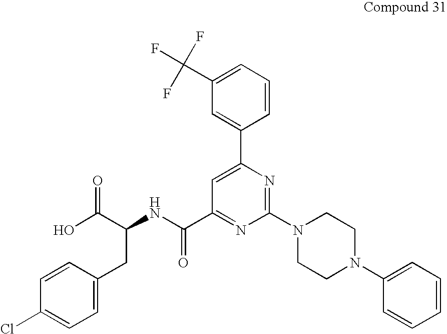

### (R)-3-(4-Chlorophenyl)-2-{[2-(4-phenyl-piperazin-1-yl)-6-(3-trifluoromethylphenyl)pyrimidine-4-carbonyl]amino}propionic acid

### Step A: 6-Chloro-2-(4-phenyl-piperazin-1-yl)pyrimidine-4-carboxylic acid methyl ester

The title compound was prepared as a minor product by a procedure analogous to that of Step A of Compound 5, by substituting 4-phenyl-piperazine for 1,2,3,4-tetrahydro isoquinoline.

### Step B: 6-Chloro-2-(4-phenyl-piperazin-1-yl)pyrimidine-4-carboxylic acid

The title compound was prepared by a procedure analogous to that of Step B of Compound 5.

### Step C: (R)-3-(4-Chlorophenyl)-2-{[6-chloro-2-(4-phenyl-piperazin-1-yl)pyrimidine-4-carbonyl]amino}propionic acid methyl ester

The title compound was prepared by a procedure analogous to that of Step C of Compound 5.

### Step D: (R)-3-(4-Chlorophenyl)-2-{[2-(4-phenyl-piperazin-1-yl)-6-(3-trifluoromethyl phenyl)pyrimidine-4-carbonyl]amino}propionic acid methyl ester

The title compound was prepared by a procedure analogous to that of Step A of Compound 6 by substituting 3-trifluoromethylphenyl boronic acid for 4-dimethylaminophenyl boronic acid.

### Step E: (R)-3-(4-Chlorophenyl)-2-{[2-(4-phenyl-piperazin-1-yl)-6-(3-trifluoromethyl phenyl)pyrimidine-4-carbonyl]amino}propionic acid

The title compound was prepared by a procedure analogous to that of Step B of Compound 6. MS 610.2 (M+H)⁺.

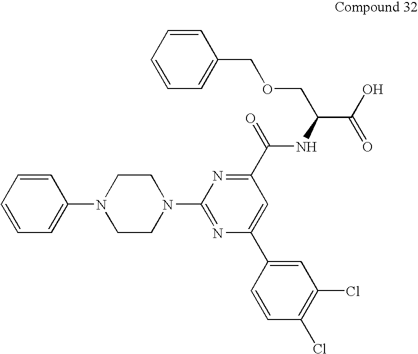

### (S)-3-Benzyloxy-2-{[6-(3,4-dichlorophenyl)-2-(4-phenyl-piperazin-1-yl)-pyrimidine-4-carbonyl]amino}propionic acid

The title compound was prepared by a procedure analogous to that of Compound 31 by substituting O-benzyl-L-serine methyl ester hydrochloride for D-4-chlorophenylalanine methyl ester hydrochloride of Step C of Compound 31, and by substituting 3,4-dichlorophenyl boronic acid for 3-trifluoromethylphenyl boronic acid of Step D of Compound 31. MS 606.2 (M+H)⁺.

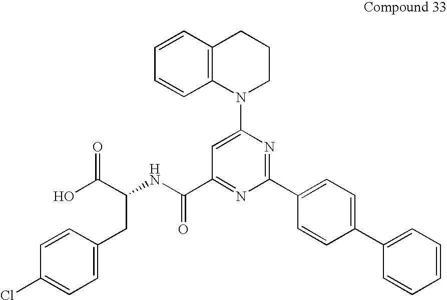

### (R)-2-{[2-Biphenyl-4-yl-6-(3,4-dihydro-2H-quinolin-1-yl)pyrimidine-4-carbonyl]amino}-3-(4-chlorophenyl)propionic acid

The title compound was prepared by procedures analogous to those of Compound 5 and Compound 6 by substituting 1,2,3,4,-tetrahydro quinoline for 1,2,3,4,-tetrahydro isoquinoline of Step A of Compound 5, and by substituting (4,4′-biphenyl)boronic acid for 4-dimethylaminophenyl boronic acid of Step A of Compound 6. MS 589.0 (M+H)⁺.

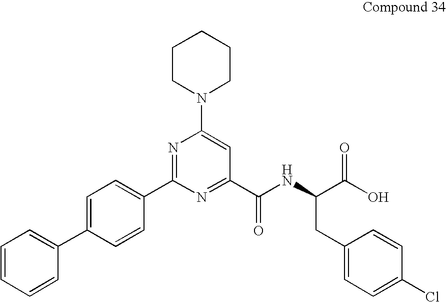

### (R)-2-[(2-Biphenyl-4-yl-6-piperidin-1-yl-pyrimidine-4-carbonyl)amino]-3-(4-chlorophenyl)propionic acid

The title compound was prepared by procedures analogous to those of Compound 5 and Compound 6 by substituting piperidine for 1,2,3,4,-tetrahydro isoquinoline of Step A of Compound 5, and by substituting (4,4′-biphenyl)boronic acid for 4-dimethylaminophenyl boronic acid of Step A of Compound 6. MS 541.0 (M+H)⁺.

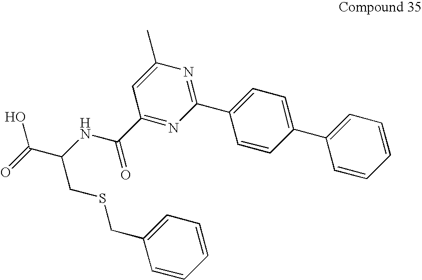

### 3-Benzylsulfanyl-2-[(2-biphenyl-4-yl-6-methyl-pyrimidine-4-carbonyl)-amino]-propionic acid

The title compound was prepared by a procedure analogous to that of Compound 1 by substituting S-benzyl-D,L-cysteine methyl ester hydrochloride for D,L-4-chlorophenylalanine ethyl ester hydrochloride of Step C of Compound 1. MS 484.1 (M+H)⁺.

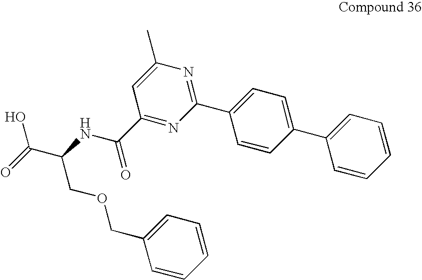

### (S)-3-Benzyloxy-2-[(2-biphenyl-4-yl-6-methyl-pyrimidine-4-carbonyl)amino]-propionic acid

The title compound was prepared by a procedure analogous to that of Compound 1 by substituting O-benzyl-L-serine methyl ester hydrochloride for D,L-4-chlorophenylalanine ethyl ester hydrochloride of Step C of Compound 1. MS 468.1 (M+H)⁺.

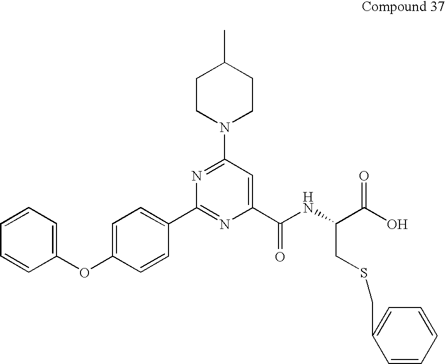

### (R)-3-Benzylsulfanyl-2-{[6-(4-methylpiperidin-1-yl)-2-(4-phenoxyphenyl)-pyrimidine-4-carbonyl]amino}propionic acid

The title compound was prepared by a procedure analogous to that of Compound 3 by substituting 4-methylpiperidine for piperidine of Step A of Compound 3, and by substituting 4-phenoxyphenylboronic acid for 4-biphenylboronic acid of Step B of Compound 3. MS 583.2 (M+H)⁺.

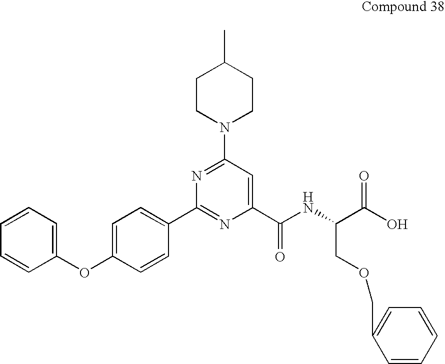

### (S)-3-Benzyloxy-2-{[6-(4-methylpiperidin-1-yl)-2-(4-phenoxyphenyl)pyrimidine-4-carbonyl]amino}propionic acid

The title compound was prepared by a procedure analogous to that of Compound 3 by substituting 4-methylpiperidine for piperidine of Step A of Compound 3, by substituting 4-phenoxyphenylboronic acid for 4-biphenylboronic acid of Step B of Compound 3, and by substituting O-benzyl-L-serine methyl ester hydrochloride for H-Cys(Bzl)-L-OMe hydrochloride of Step D of Compound 3. MS 567.2 (M+H)⁺.

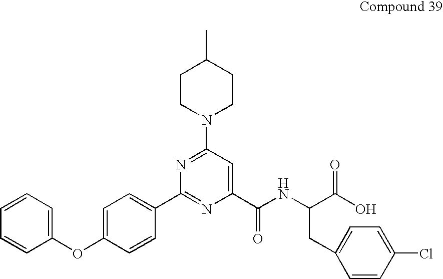

### 3-(4-Chlorophenyl)-2-{[6-(4-methylpiperidin-1-yl)-2-(4-phenoxyphenyl)-pyrimidine-4-carbonyl]amino}propionic acid

The title compound was prepared by a procedure analogous to that of Compound 3 by substituting 4-methylpiperidine for piperidine of Step A of Compound 3, by substituting 4-phenoxyphenylboronic acid for 4-biphenylboronic acid of Step B of Compound 3, and by substituting D,L-4-chlorophenylalanine ethyl ester hydrochloride for H-Cys(Bzl)-L-OMe hydrochloride of Step D of Compound 3. MS 571.0 (M+H)⁺.

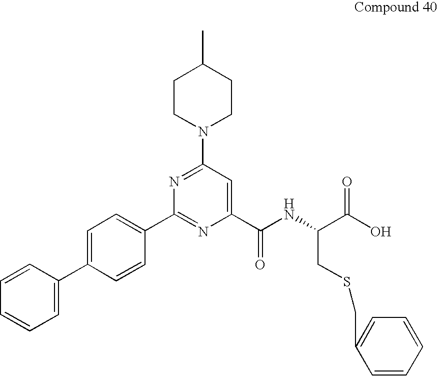

### (R)-3-Benzylsulfanyl-2-{[2-biphenyl-4-yl-6-(4-methylpiperidin-1-yl)pyrimidine-4-carbonyl]amino}propionic acid

The title compound was prepared by a procedure analogous to that of Compound 3 by substituting 4-methylpiperidine for piperidine of Step A of Compound 3. MS 567.2 (M+H)⁺.

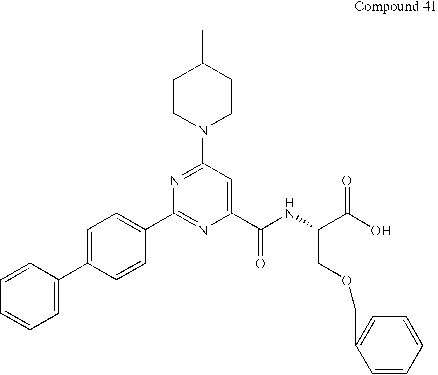

### (S)-3-Benzyloxy-2-{[2-biphenyl-4-yl-6-(4-methylpiperidin-1-yl)pyrimidine-4-carbonyl]amino}propionic acid

The title compound was prepared by a procedure analogous to that of Compound 3 by substituting 4-methylpiperidine for piperidine of Step A of Compound 3, and by substituting O-benzyl-L-serine methyl ester hydrochloride for H-Cys(Bzl)-L-OMe hydrochloride of Step D of Compound 3. MS 551.1 (M+H)⁺.

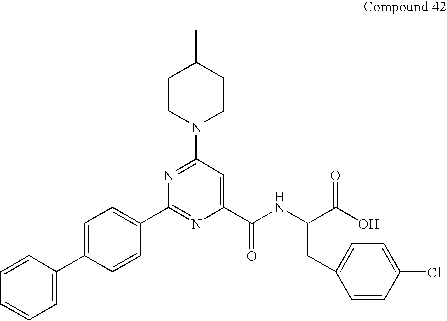

### 2-{[2-Biphenyl-4-yl-6-(4-methylpiperidin-1-yl)pyrimidine-4-carbonyl]amino}-3-(4-chlorophenyl)propionic acid

The title compound was prepared by a procedure analogous to that of Compound 3 by substituting 4-methylpiperidine for piperidine of Step A of Compound 3, and by substituting D,L-4-chlorophenylalanine ethyl ester hydrochloride for H-Cys(Bzl)-L-OMe hydrochloride of Step D of Compound 3. MS 555.2 (M+H)⁺.

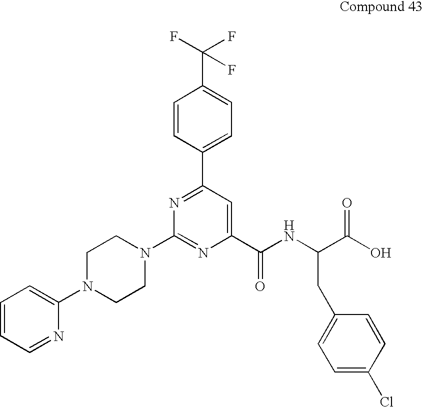

### 3-(4-Chlorophenyl)-2-{[2-(4-pyridin-2-yl-piperazin-1-yl)-6-(4-trifluoromethyl-phenyl)pyrimidine-4-carbonyl]amino}propionic acid

The title compound was prepared by a procedure analogous to that of Compound 31 by substituting 1-pyridin-2-yl-piperazine for 4-phenyl-piperazine of Step A of Compound 31, by substituting D,L-4-chlorophenylalanine ethyl ester hydrochloride for D-4-chlorophenylalanine methyl ester hydrochloride of Step C of Compound 31, and by substituting 4-trifluoromethylphenyl boronic acid for 3-trifluoromethylphenyl boronic acid of Step D of Compound 31. MS 611.1 (M+H)⁺.

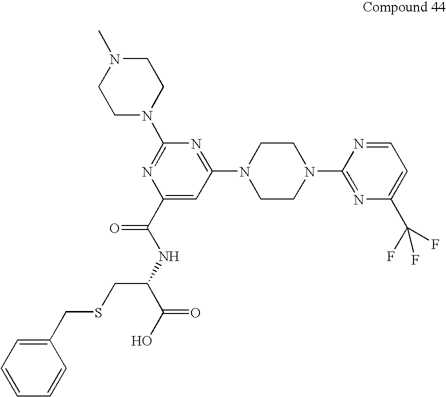

### (R)-3-Benzylsulfanyl-2-({2-(4-methyl-piperazin-1-yl)-6-[4-(4-trifluoromethyl-pyrimidin-2-yl)piperazin-1-yl]pyrimidine-4-carbonyl}amino)propionic acid

The title compound was prepared by a procedure analogous to that of Compound 4 by substituting 2-piperazin-1-yl-4-trifluoromethyl-pyrimidine for piperidine of Step A of Compound 4, by substituting 1-methyl-piperazine for 4-phenylpiperidine of Step B of Compound 4, and by substituting H-Cys(Bzl)-L-OMe hydrochloride for D-4-chlorophenylalanine methyl ester hydrochloride of step D of Compound 4. MS 646.1 (M+H)⁺.

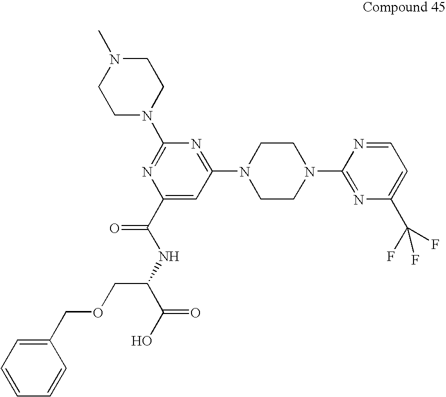

### (S)-3-Benzyloxy-2-({2-(4-methyl-piperazin-1-yl)-6-[4-(4-trifluoromethylpyrimidin-2-yl)piperazin-1-yl]pyrimidine-4-carbonyl}amino)propionic acid

The title compound was prepared by a procedure analogous to that of Compound 4 by substituting 2-piperazin-1-yl-4-trifluoromethyl-pyrimidine for piperidine of Step A of Compound 4, by substituting 1-methyl-piperazine for 4-phenylpiperidine of Step B of Compound 4, and by substituting O-benzyl-L-serine methyl ester hydrochloride for D-4-chlorophenylalanine methyl ester hydrochloride of step D of Compound 4. MS 630.2 (M+H)⁺.

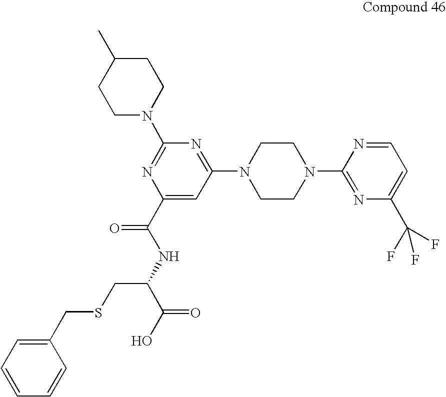

### (R)-3-Benzylsulfanyl-2-({2-(4-methylpiperidin-1-yl)-6-[4-(4-trifluoromethyl-pyrimidin-2-yl)piperazin-1-yl]pyrimidine-4-carbonyl}amino)propionic acid

The title compound was prepared by a procedure analogous to that of Compound 4 by substituting 2-piperazin-1-yl-4-trifluoromethyl-pyrimidine for piperidine of Step A of Compound 4, by substituting 4-methylpiperidine for 4-phenylpiperidine of Step B of Compound 4, and by substituting H-Cys(Bzl)-L-OMe hydrochloride for D-4-chlorophenylalanine methyl ester hydrochloride of step D of Compound 4. MS 645.2 (M+H)⁺.

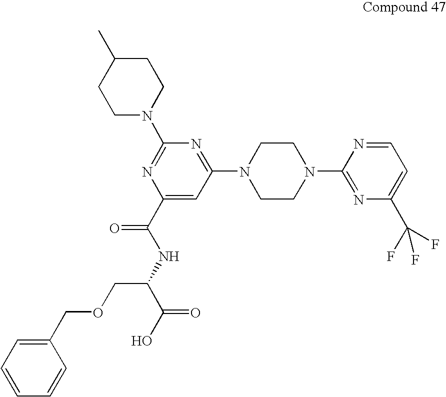

### (S)-3-Benzyloxy-2-({2-(4-methylpiperidin-1-yl)-6-[4-(4-trifluoromethylpyrimidin-2-yl)piperazin-1-yl]pyrimidine-4-carbonyl}amino)propionic acid

The title compound was prepared by a procedure analogous to that of Compound 4 by substituting 2-piperazin-1-yl-4-trifluoromethyl-pyrimidine for piperidine of Step A of Compound 4, by substituting 4-methylpiperidine for 4-phenylpiperidine of Step B of Compound 4, and by substituting O-benzyl-L-serine methyl ester hydrochloride for D-4-chlorophenylalanine methyl ester hydrochloride of step D of Compound 4. MS 629.2 (M+H)⁺.

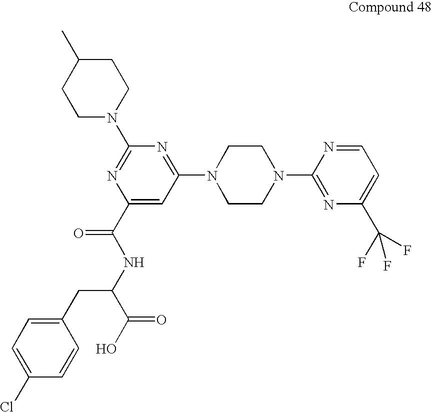

### 3-(4-Chlorophenyl)-2-({2-(4-methylpiperidin-1-yl)-6-[4-(4-trifluoromethyl-pyrimidin-yl)piperazin-1-yl]pyrimidine-4-carbonyl}amino)propionic acid

The title compound was prepared by a procedure analogous to that of Compound 4 by substituting 2-piperazin-1-yl-4-trifluoromethyl-pyrimidine for piperidine of Step A of Compound 4, by substituting 4-methylpiperidine for 4-phenylpiperidine of Step B of Compound 4, and by substituting D,L-4-chlorophenylalanine ethyl ester hydrochloride for D-4-chlorophenylalanine methyl ester hydrochloride of step D of Compound 4. MS 633.2 (M+H)⁺.

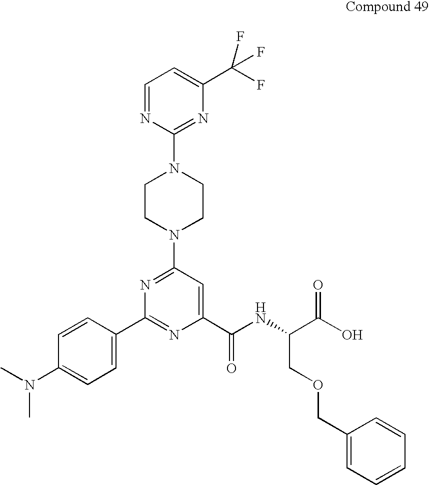

### (S)-3-Benzyloxy-2-({2-(4-dimethylaminophenyl)-6-[4-(4-trifluoromethylpyrimidin-2-yl)piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, by substituting 4-dimethylaminophenyl boronic acid for 4-biphenylboronic acid of Step B of Compound 3, and by substituting O-benzyl-L-serine methyl ester hydrochloride for H-Cys(Bzl)-L-OMe hydrochloride of Step D of Compound 3. MS 651.2 (M+H)⁺.

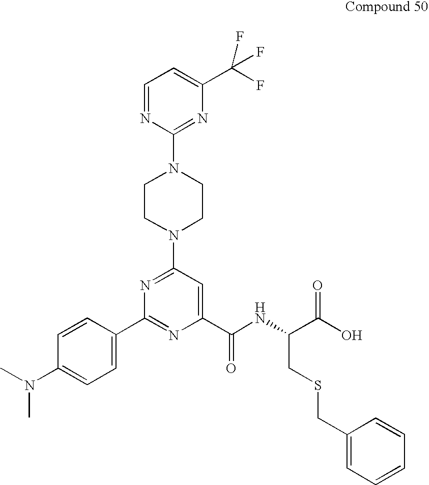

### (R)-3-Benzylsulfanyl-2-({2-(4-dimethylaminophenyl)-6-[4-(4-trifluoromethyl-pyrimidin-2-yl)piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, and by substituting 4-dimethylaminophenyl boronic acid for 4-biphenylboronic acid of Step B of Compound 3. MS 667.2 (M+H)⁺.

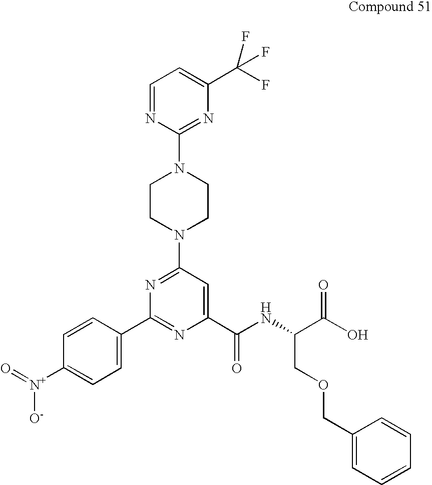

### (S)-3-Benzyloxy-2-({2-(4-nitrophenyl)-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, by substituting 4-nitrophenyl boronic acid for 4-biphenylboronic acid of Step B of Compound 3, and by substituting O-benzyl-L-serine methyl ester hydrochloride for H-Cys(Bzl)-L-OMe hydrochloride of Step D of Compound 3. MS 653.2 (M+H)⁺.

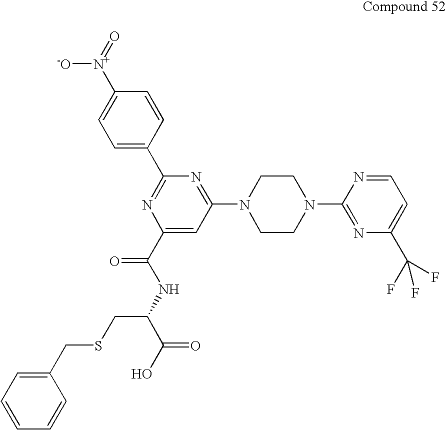

### (R)-3-Benzylsulfanyl-2-({2-(4-nitrophenyl)-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, and by substituting 4-nitrophenyl boronic acid for 4-biphenylboronic acid of Step B of Compound 3. MS 669.2 (M+H)⁺.

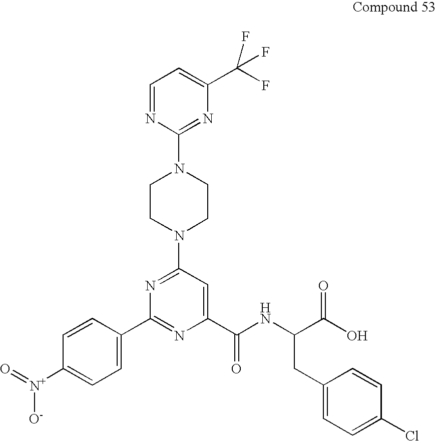

### 3-(4-Chlorophenyl)-2-({2-(4-nitrophenyl)-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, by substituting 4-nitrophenyl boronic acid for 4-biphenylboronic acid of Step B of Compound 3, and by substituting D,L-chlorophenylalanine ethyl ester hydrochloride for H-Cys(Bzl)-L-OMe hydrochloride of Step D of Compound 3. MS 657.0 (M+H)⁺.

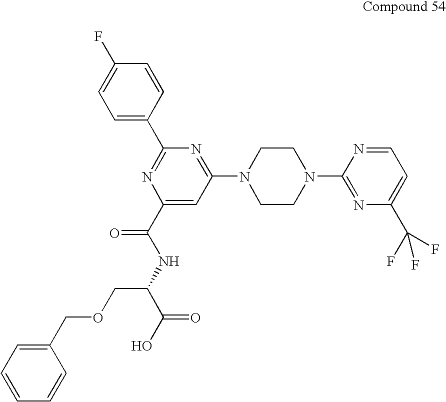

### (S)-3-Benzyloxy-2-({2-(4-fluorophenyl)-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, by substituting 4-fluorophenylboronic acid for 4-biphenylboronic acid of Step B of Compound 3, and by substituting O-benzyl-L-serine methyl ester hydrochloride for H-Cys(Bzl)-L-OMe hydrochloride of Step D of Compound 3. MS 626.1 (M+H)⁺.

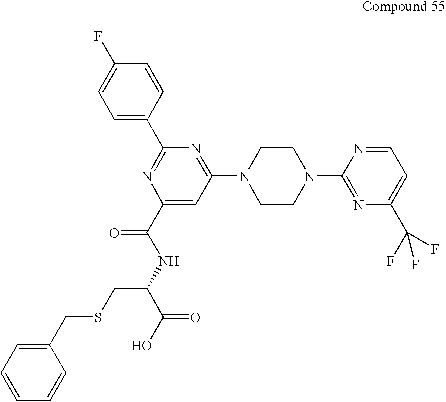

### (R)-3-Benzylsulfanyl-2-({2-(4-fluorophenyl)-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, and by substituting 4-fluorophenylboronic acid for 4-biphenylboronic acid of Step B of Compound 3; MS 642.0 (M+H)⁺.

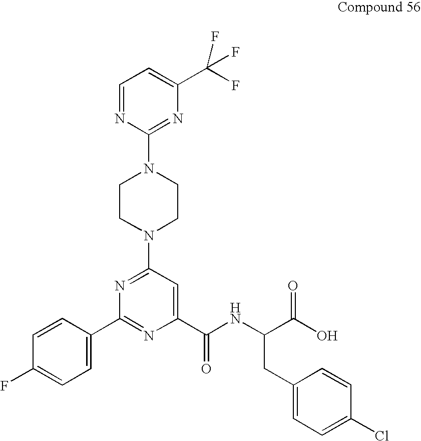

### 3-(4-Chlorophenyl)-2-({2-(4-fluorophenyl)-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, by substituting 4-fluorophenylboronic acid for 4-biphenylboronic acid of Step B of Compound 3, and by substituting D,L-4-chlorophenylalanine ethyl ester hydrochloride for H-Cys(Bzl)-L-OMe hydrochloride of Step D of Compound 3. MS 630.0 (M+H)⁺.

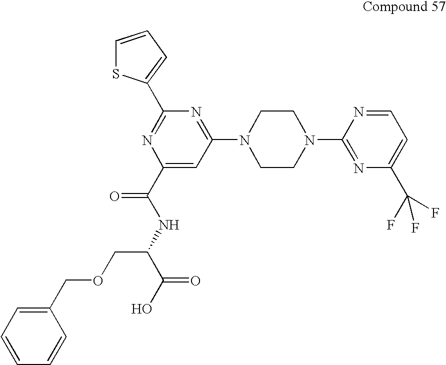

### (S)-3-Benzyloxy-2-({2-thiophen-2-yl-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, by substituting 2-thiophene boronic acid for 4-biphenylboronic acid of Step B of Compound 3, and by substituting O-benzyl-L-serine methyl ester hydrochloride for H-Cys(Bzl)-L-OMe hydrochloride of Step D of Compound 3. MS 614.1 (M+H)⁺.

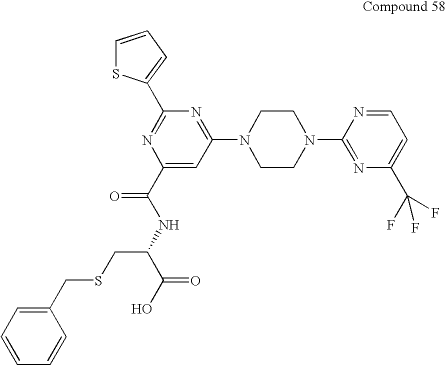

### (R)-3-Benzylsulfanyl-2-({2-thiophen-2-yl-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, and by substituting 2-thiophene boronic acid for 4-biphenylboronic acid of Step B of Compound 3. MS 630.0 (M+H)⁺.

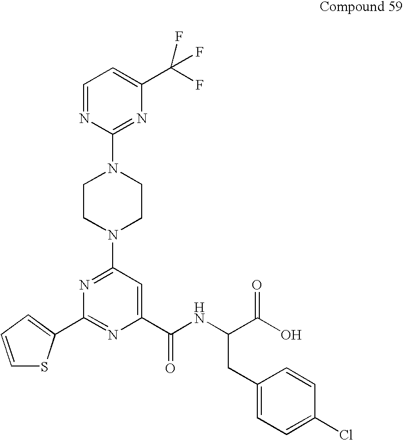

### 3-(4-Chlorophenyl)-2-({2-thiophen-2-yl-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, by substituting 2-thiophene boronic acid for 4-biphenylboronic acid of Step B of Compound 3, and by substituting D,L-4-chlorophenylalanine ethyl ester hydrochloride for H-Cys(Bzl)-L-OMe hydrochloride of Step D of Compound 3. MS 618.0 (M+H)⁺.

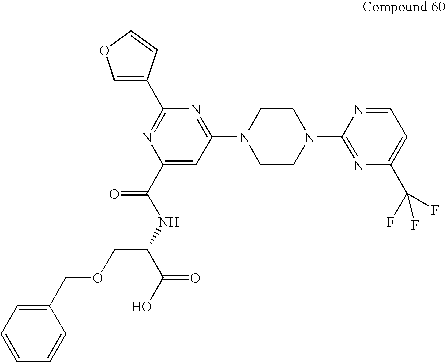

### (S)-3-Benzyloxy-2-({2-furan-3-yl-6-[4-(4-trifluoromethylpyrimidin-2-yl)piperazin-1yl]-pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, by substituting 3-furan boronic acid for 4-biphenylboronic acid of Step B of Compound 3, and by substituting O-benzyl-L-serine methyl ester hydrochloride for H-Cys(Bzl)-L-OMe hydrochloride of Step D of Compound 3. MS 598.0 (M+H)⁺.

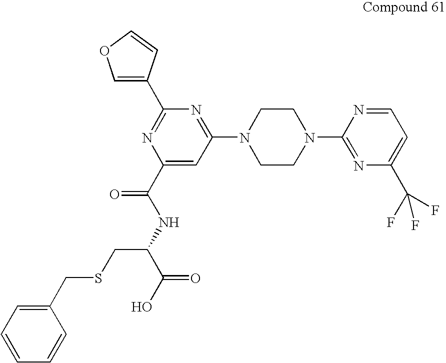

### (R)-3-Benzylsulfanyl-2-({2-furan-3-yl-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, and by substituting 3-furan boronic acid for 4-biphenylboronic acid of Step B of Compound 3. MS 614.1 (M+H)⁺.

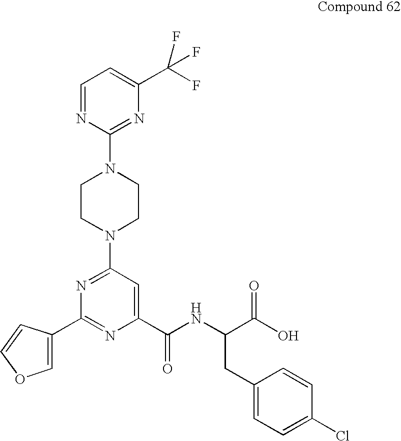

### 3-(4-Chlorophenyl)-2-({2-furan-3-yl-6-[4-(4-trifluoromethylpyrimidin-2-yl)-piperazin-1-yl]pyrimidine-4-carbonyl}amino)propionic acid

The title compound was prepared by a procedure analogous to that of Compound 3 by substituting 2-piperazin-1-yl-4-trifluoromethyl-pyrimidine for piperidine of Step A of Compound 3, by substituting 3-furan boronic acid for 4-biphenylboronic acid of Step B of Compound 3, and by substituting D,L-4-chlorophenylalanine ethyl ester hydrochloride for H-Cys(Bzl)-L-OMe hydrochloride of Step D of Compound 3. MS 602.0 (M+H)⁺.

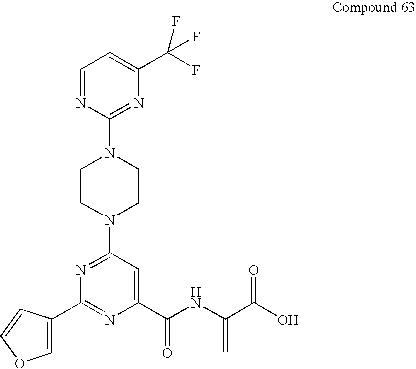

### 2-({2-Furan-3-yl-6-[4-(4-trifluoromethylpyrimidin-2-yl)piperazin-1-yl]pyrimidine-4-carbonyl}amino)acrylic acid

The title compound was prepared by a procedure analogous to that of Compound 3 by substituting 2-piperazin-1-yl-4-trifluoromethyl-pyrimidine for piperidine of Step A of Compound 3, by substituting 3-furan boronic acid for 4-biphenylboronic acid of Step B of Compound 3. In this Compound the sulfide compound was oxidized to the corresponding sulfone derivative, which underwent elimination during workup to give the title compound. MS 490.2 (M+H)⁺.

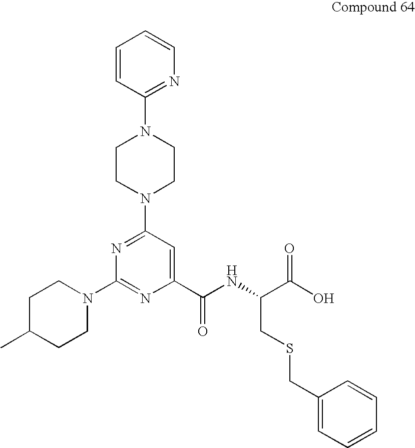

### (R)-3-Benzylsulfanyl-2-{[2-(4-methylpiperidin-1-yl)-6-(⁴-pyridin-2-yl-piperazin-1-yl)pyrimidine-4-carbonyl]amino}propionic acid

The title compound was prepared by a procedure analogous to that of Compound 4 by substituting 1-pyridin-2-yl-piperazine for piperidine of Step A of Compound 4, by substituting 4-methylpiperidine for 4-phenylpiperidine of Step B of Compound 4, and by substituting H-Cys(Bzl)-L-OMe hydrochloride for D-4-chlorophenylalanine methyl ester hydrochloride of Step D of Compound 4. MS 576.3 (M+H)⁺.

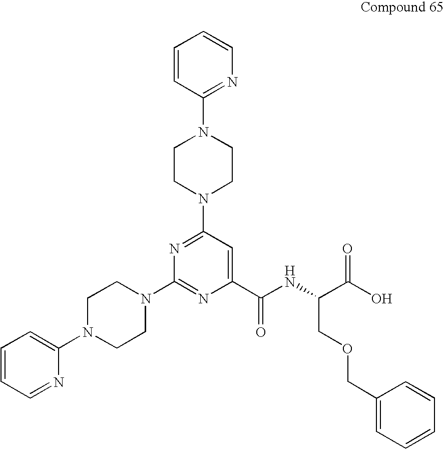

### (S)-3-Benzyloxy-2-{[2,6-bis-(4-pyridin-2-yl-piperazin-1-yl)pyrimidine-4-carbonyl]-amino}propionic acid

The title compound was prepared by a procedure analogous to that of Compound 4 by substituting 1-pyridin-2-yl-piperazine for piperidine of Step A of Compound 4, by substituting 1-pyridin-2-yl-piperazine for 4-phenylpiperidine of Step B of Compound 4, and by substituting O-benzyl-L-serine methyl ester hydrochloride for D-4-chlorophenylalanine methyl ester hydrochloride of step D of Compound 4. MS 624.2 (M+H)⁺.

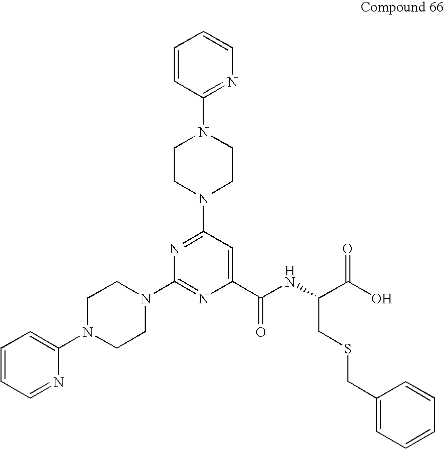

### (R)-3-Benzylsulfanyl-2-{[2,6-bis-(4-pyridin-2-yl-piperazin-1-yl)pyrimidine-4-carbonyl]amino}propionic acid

The title compound was prepared by a procedure analogous to that of Compound 4 by substituting 1-pyridin-2-yl-piperazine for piperidine of Step A of Compound 4, by substituting 1-pyridin-2-yl-piperazine for 4-phenylpiperidine of Step B of Compound 4, and by substituting H-Cys(Bzl)-L-OMe hydrochloride for D-4-chlorophenylalanine methyl ester hydrochloride of step D of Compound 4. MS 640.2 (M+H)⁺.

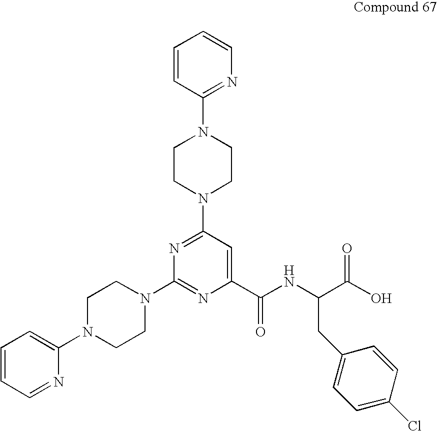

### 2-{[2,6-Bis-(4-pyridin-2-yl-piperazin-1-yl)pyrimidine-4-carbonyl]amino}-3-(4-chlorophenyl)propionic acid

The title compound was prepared by a procedure analogous to that of Compound 4 by substituting 1-pyridin-2-yl-piperazine for piperidine of Step A of Compound 4, by substituting 1-pyridin-2-yl-piperazine for 4-phenylpiperidine of Step B of Compound 4, and by substituting D,L-4-chlorophenylalanine ethyl ester hydrochloride for D-4-chlorophenylalanine methyl ester hydrochloride of step D of Compound 4. MS 628.2 (M+H)⁺.

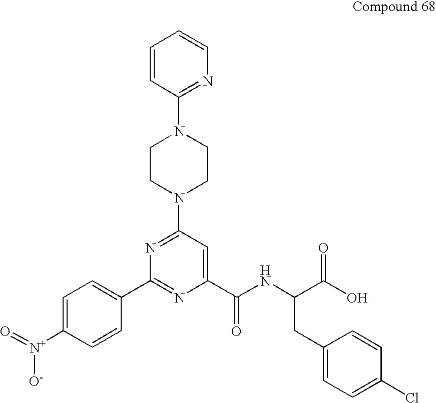

### 3-(4-Chlorophenyl)-2-{[2-(4-nitrophenyl)-6-(4-pyridin-2-yl-piperazin-1-yl)-pyrimidine-4-carbonyl]amino}propionic acid

The title compound was prepared by a procedure analogous to that of Compound 3 by substituting 1-pyridin-2-yl-piperazine for piperidine of Step A of Compound 3 by substituting 4-nitrophenyl boronic acid for 4-biphenylboronic acid of Step B of Compound 3 and by substituting D,L-4-chlorophenylalanine ethyl ester hydrochloride for H-Cys(Bzl)-L-OMe hydrochloride of Step D of Compound 3. MS 589.0 (M+H)⁺.

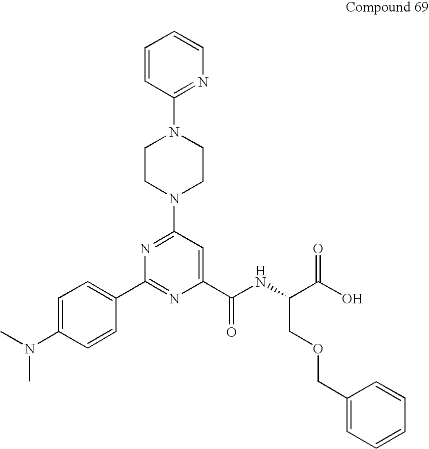

### (S)-3-Benzyloxy-2-{[2-(4-dimethylaminophenyl)-6-(4-pyridin-2-yl-piperazin-1-yl)-pyrimidine-4-carbonyl]amino}propionic acid

The title compound was prepared by a procedure analogous to that of Compound 3 by substituting 1-pyridin-2-yl-piperazine for piperidine of Step A of Compound 3, by substituting 4-dimethylaminophenyl boronic acid for 4-biphenylboronic acid of Step B of Compound 3, and by substituting O-benzyl-L-serine methyl ester hydrochloride for H-Cys(Bzl)-L-OMe hydrochloride of Step D of Compound 3. MS 582.2 (M+H)⁺.

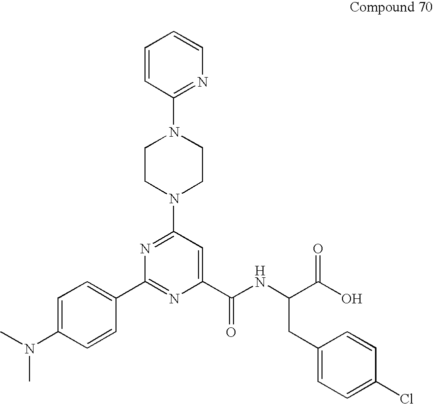

### 3-(4-Chlorophenyl)-2-{[2-(4-dimethylaminophenyl)-6-(4-pyridin-2-yl-piperazin-1-yl)pyrimidine-4-carbonyl]amino}propionic acid

The title compound was prepared by a procedure analogous to that of Compound 3 by substituting 1-pyridin-2-yl-piperazine for piperidine of Step A of Compound 3, by substituting 4-dimethylaminophenyl boronic acid for 4-biphenylboronic acid of Step B of Compound 3, and by substituting D,L-4-chlorophenylalanine ethyl ester hydrochloride for H-Cys(Bzl)-L-OMe hydrochloride of Step D of Compound 3. MS 586.0 (M+H)⁺.

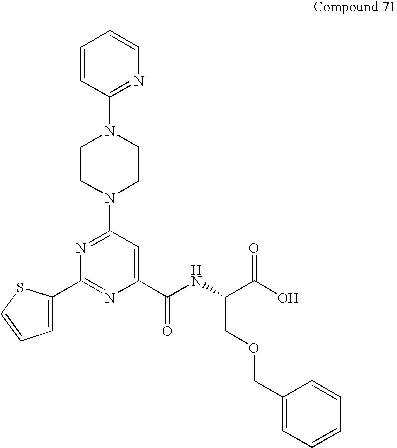

### (S)-3-Benzyloxy-2-{[6-(4-pyridin-2-yl-piperazin-1-yl)-2-thiophen-2-yl-pyrimidine-4-carbonyl]amino}propionic acid

The title compound was prepared by a procedure analogous to that of Compound 3 by substituting 1-pyridin-2-yl-piperazine for piperidine of Step A of Compound 3, by substituting 2-thiophene boronic acid for 4-biphenylboronic acid of Step B of Compound 3, and by substituting O-benzyl-L-serine methyl ester hydrochloride for H-Cys(Bzl)-L-OMe hydrochloride of Step D of Compound 3. MS 545.1 (M+H)⁺.

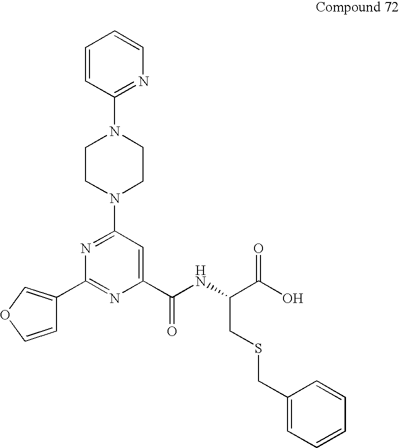

### (R)-3-Benzylsulfanyl-2-{[2-furan-3-yl-6-(4-pyridin-2-yl-piperazin-1-yl)pyrimidine-4-carbonyl]amino}propionic acid

The title compound was prepared by a procedure analogous to that of Compound 3 by substituting 1-pyridin-2-yl-piperazine for piperidine of Step A of Compound 3, and by substituting 3-furan boronic acid for 4-biphenylboronic acid of Step B of Compound 3. MS 545.1 (M+H)⁺.

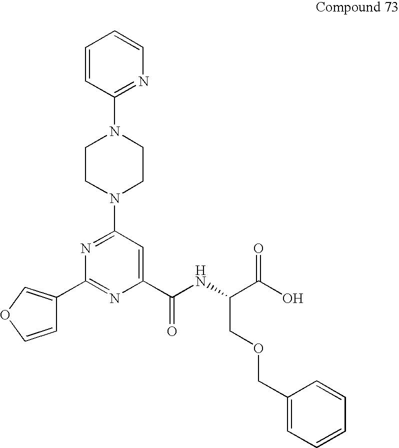

### (S)-3-Benzyloxy-2-{[2-furan-3-yl-6-(4-pyridin-2-yl-piperazin-1-yl)pyrimidine-4-carbonyl]amino}propionic acid

The title compound was prepared by a procedure analogous to that of Compound 3 by substituting 1-pyridin-2-yl-piperazine for piperidine of Step A of Compound 3, by substituting 3-furan boronic acid for 4-biphenylboronic acid of Step B of Compound 3, and by substituting O-benzyl-L-serine methyl ester hydrochloride for H-Cys(Bzl)-L-OMe hydrochloride of Step D of Compound 3. MS 529.2 (M+H)⁺.

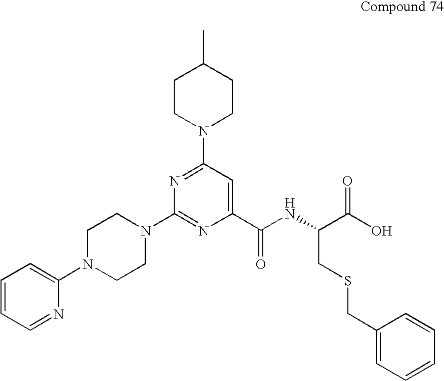

### (R)-3-Benzylsulfanyl-2-{[6-(4-methylpiperidin-1-yl)-2-(4-pyridin-2-yl-piperazin-1-yl)pyrimidine-4-carbonyl]amino}propionic acid

The title compound was prepared by a procedure analogous to that of Compound 4 by substituting 4-methylpiperidine for piperidine of Step A of Compound 4, by substituting 1-pyridin-2-yl-piperazine for 4-phenylpiperidine of Step B of Compound 4, and by substituting H-Cys(Bzl)-L-OMe hydrochloride for D-4-chlorophenylalanine methyl ester hydrochloride of step D of Compound 4. MS 576.2 (M+H)⁺.

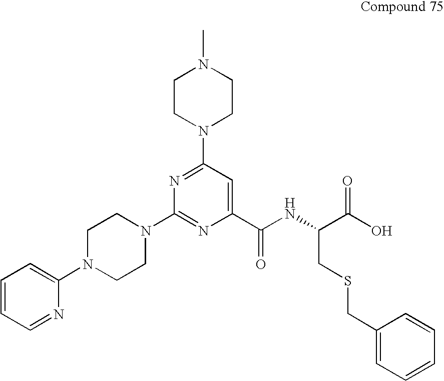

### (R)-3-Benzylsulfanyl-2-{[6-(4-methyl-piperazin-1-yl)-2-(4-pyridin-2-yl-piperazin-1-yl)pyrimidine-4-carbonyl]amino}propionic acid

The title compound was prepared by a procedure analogous to that of Compound 4 by substituting 1-methyl-piperazine for piperidine of Step A of Compound 4, by substituting 1-pyridin-2-yl-piperazine for 4-phenylpiperidine of Step B of Compound 4, and by substituting H-Cys(Bzl)-L-OMe hydrochloride for D-4-chlorophenylalanine methyl ester hydrochloride of step D of Compound 4. MS 577.2 (M+H)⁺.

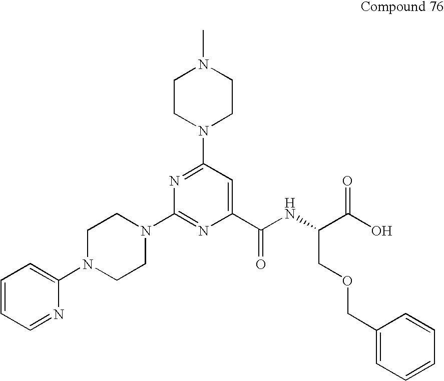

### (S)-3-Benzyloxy-2-{[6-(4-methyl-piperazin-1-yl)-2-(4-pyridin-2-yl-piperazin-1-yl)-pyrimidine-4-carbonyl]amino}propionic acid

The title compound was prepared by a procedure analogous to that of Compound 4 by substituting 1-methyl-piperazine for piperidine of Step A of Compound 4, by substituting 1-pyridin-2-yl-piperazine for 4-phenylpiperidine of Step B of Compound 4, and by substituting O-Benzyl-L-serine methyl ester hydrochloride for D-4-chlorophenylalanine methyl ester hydrochloride of step D of Compound 4. MS 561.2 (M+H)⁺.

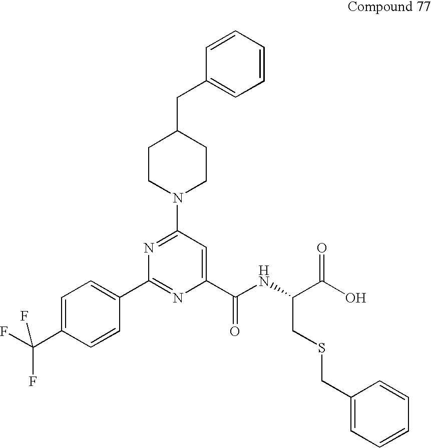

### (R)-2-{[6-(4-Benzylpiperidin-1-yl)-2-(4-trifluoromethylphenyl)pyrimidine-4-carbonyl]amino}3-benzylsulfanylpropionic acid

The title compound was prepared by a procedure analogous to that of Compound 3 by substituting 4-benzylpiperidine for piperidine of Step A of Compound 3, and by substituting 4-trifluoromethylphenyl boronic acid for 4-biphenylboronic acid of Step B of Compound 3. MS 635.1 (M+H)⁺.

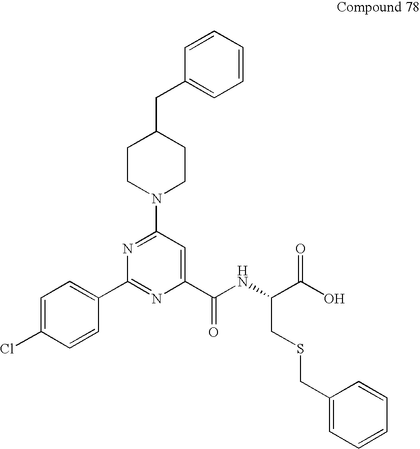

### (R)-2-{[6-(4-Benzylpiperidin-1-yl)-2-(4-chlorophenyl)pyrimidine-4-carbonyl]-amino}-3-benzylsulfanylpropionic acid

The title compound was prepared by a procedure analogous to that of Compound 3 by substituting 4-benzylpiperidine for piperidine of Step A of Compound 3, and by substituting 4-chlorophenyl boronic acid for 4-biphenylboronic acid of Step B of Compound 3. MS 601.0 (M+H)⁺.

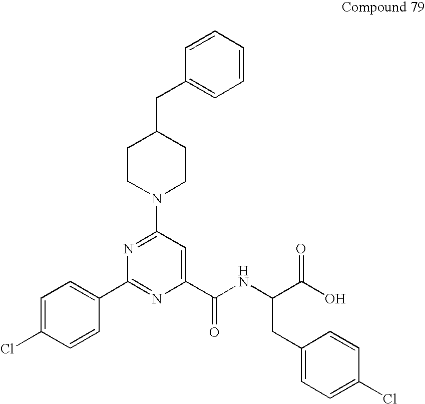

### 2-{[6-(4-Benzylpiperidin-1-yl)-2-(4-chlorophenyl)pyrimidine-4-carbonyl]amino}-3-(4-chlorophenyl)propionic acid

The title compound was prepared by a procedure analogous to that of Compound 3 by substituting 4-benzylpiperidine for piperidine of Step A of Compound 3, by substituting 4-chlorophenyl boronic acid for 4-biphenylboronic acid of Step B of Compound 3, and by substituting D,L-4-chlorophenylalanine ethyl ester hydrochloride for H-Cys(Bzl)-L-OMe hydrochloride of Step D of Compound 3. MS 589.0 (M+H)⁺.

### Example 2

### Assay to Evaluate Effect on Type III Protein Secretion Systems

The ability of the compounds of the invention to inhibit Type III protein secretion systems may be analyzed as follows.

Primary assay: Type III protein secretion of the chimeric SopE′-′Bla polypeptide by Salmonella enterica. This procedure is a cell-based assay that measures the type III-dependent secretion by Salmonella enterica of a plasmid-encoded chimeric polypeptide whose synthesis can be regulated, and which is endowed with an enzymatic activity that can be monitored calorimetrically by hydrolysis of a substrate that is unable to penetrate into the bacterial cytoplasm within the time constraints of the reaction. Thus, the colorimetric reaction is not influenced by SopE′-′Bla polypeptide in the bacterial cytoplasm. Instead, it effectively measures the amount of polypeptide that has been secreted from the S. enterica cytoplasm to the extracellular medium via type III system protein secretion.

The SopE′-′Bla recombinant polypeptide consists of two functionally distinct domains spliced together. The N-terminus domain is encoded by a polynucleotide region specifying the signal for type III secretion of the SopE polypeptide of S. enterica, an effector of the SPI1 type III protein secretion system. The C-terminus domain of SopE′-′Bla consists of a 263 amino acid peptide sequence that corresponds to the TEM-1 β-lactamase expressed by plasmid pBR322 but without its N-terminal signal sequence. The TEM-1 β-lactamase part of the SopE′-′Bla chimeric polypeptide is used as a reporter enzyme. It is capable of hydrolyzing nitrocefin resulting in a product whose accumulation can be monitored by colorimetric detection. The secretion of the SopE′-′Bla chimeric polypeptide from the cytoplasm to the extracellular medium is dependent on type III protein secretion.

For this procedure, cells grown under conditions known to favor a functional SPI1 secretion system are induced for expression of the SopE′-′Bla protein and grown either in the presence or in the absence of putative inhibitors for determined time. Nitrocefin is then added to the various cultures and its hydrolysis are used for quantitation. An inhibitor of Type III protein secretion is generally a compound that reduces the signal of the enzymatic reaction by decreasing the amount of SopE′-′Bla secreted into the extracellular medium.

Secondary assay: Type III-dependent protein secretion of the SipB polypeptide by S. enterica. The SipB protein of S. enterica is another effector of the SPI1 type III protein secretion system from S. enterica. In this cell-based procedure, the Type III-dependent secretion of SipB from the bacterial cytoplasm to the extracellular medium was measured through its reactivity with a cognate mouse monoclonal.

Salmonella enterica cells growing either in the presence or in the absence of inhibitors are induced for the production of SipB. Following an established period of growth the cells are sedimented and the amount of SipB present in the supernatant is quantified with a scanning imager following application of immunoblot techniques. Detection may employ an anti-SipB mouse monoclonal antibody (e.g., obtained from Jorge Galan, SUNY at Stony Brook, N.Y.) followed by treatment with commercially available sheep anti-mouse polyclonal antibody conjugated with horseradish peroxidase. Thereafter the membrane is treated with a peroxidase chemiluminescent substrate and exposed to film for an appropriate exposure time. Inhibition may be measured relative to untreated controls.

Tertiary assay: inhibition of Type III protein secretion of effectors from a Pseudomonas aeruginosa system. Type III protein secretion is used by P. aeruginosa to secrete several essential virulence determinants. One effector of the type III protein secretion system of P. aeruginosa PA 103 is the virulence determinants ExoU.

The amount of Type III-dependent secretion of ExoU by P. aeruginosa PA103 can be determined in a cell-based assay by quantification of the 73.9 kDa ExoU protein secreted into the extracellular medium. Such quantitation can be achieved by growing strain PA 103 in a deferrated medium in the presence of nitrilotriacetic acid (an inducer of Type III protein secretion in P. aeruginosa) and either in the presence or absence of putative inhibitors. After a prolonged growth period, the cells are sedimented and the supernatants concentrated by ammonium sulfate precipitation. The proteins in the resuspended pellets are separated by electrophoresis on SDS-polyacrylamide gels. After staining gels with Colloidal Blue™, the 73.9 kDa ExoU band is quantitated by scanning through an imager. The effects of inhibitors on the intensity of the ExoU band may be measured relative to that of untreated controls.

By way of example, assay results for preferred compounds of the invention are provided below in Table I.

TABLE I

| Compound | ExoU IC50(uM) | SipB IC50(uM) | SopE IC50(uM) |
|---|---|---|---|
| 1 | 83.9 | 4.40 | 7.40 |
| 2 | >100 | | 34.20 |
| 3 | 37.6 | 3.00 | 4.10 |
| 4 | >100 | 27.04 | 2.00 |
| 5 | | | 32.70 |
| 6 | 68.4 | 50.00 | 4.50 |
| 7 | | >100 | 1.20 |
| 8 | | >100 | 2.00 |
| 9 | | | 8.00 |
| 10 | | >100 | 4.30 |
| 11 | 92.2 | 44.20 | 1.40 |
| 12 | >100 | 28.10 | 1.40 |
| 13 | >100 | 19.40 | 1.30 |
| 14 | >100 | 27.60 | 1.80 |
| 15 | 68.1 | 8.50 | 3.60 |
| 16 | 62.2 | 10.20 | 6.20 |
| 17 | >100 | 23.00 | 1.60 |
| 18 | 46.6 | 16.80 | 19.00 |
| 19 | 67.8 | 8.00 | 6.40 |
| 20 | | >100 | 2.10 |
| 21 | 39.0 | 24.80 | 3.60 |
| 22 | 52.6 | >100 | 1.80 |
| 23 | 73.0 | 31.70 | 3.30 |
| 24 | 46.9 | 39.20 | 5.45 |
| 25 | 57.2 | 51.90 | 6.50 |
| 26 | >100 | 46.80 | 50.10 |
| 27 | >100 | 8.80 | 5.30 |
| 28 | | 18.50 | 5.20 |
| 29 | | | 3.20 |
| 30 | >100 | 17.90 | 4.60 |
| 31 | 88.7 | 13.30 | 4.10 |
| 32 | 40.7 | 5.70 | 3.20 |
| 33 | 24.4 | | |
| 34 | 71.0 | | |
| 35 | >100 | 59.20 | 17.70 |
| 36 | >100 | 79.60 | 32.70 |
| 37 | | >100 | 1.70 |
| 38 | 82.8 | 7.10 | 1.30 |
| 39 | 74.5 | 13.16 | 1.50 |
| 40 | 61.2 | 23.04 | 5.10 |
| 41 | 67.4 | 28.60 | 2.40 |
| 42 | >100 | 22.30 | 4.80 |
| 43 | >100 | 74.20 | 8.10 |
| 44 | | >100 | 85.00 |
| 45 | | | 60.70 |
| 46 | | 10.60 | 3.40 |
| 47 | >100 | 10.40 | 4.50 |
| 48 | >100 | 40.00 | 1.30 |
| 49 | 92.6 | 7.20 | 5.90 |
| 50 | | >100 | 20.10 |
| 51 | 55.0 | 84.50 | 1.90 |
| 52 | | 15.00 | 10.40 |
| 53 | 40.4 | 84.60 | 1.20 |
| 54 | | 2.70 | 1.30 |
| 55 | | 20.50 | 2.40 |
| 56 | 55.4 | 4.50 | 2.20 |
| 57 | | 13.30 | 28.70 |
| 58 | | 10.60 | 4.50 |
| 59 | | 17.10 | 1.80 |
| 60 | >100 | 15.60 | 16.40 |
| 61 | >100 | 43.20 | 10.20 |
| 62 | | 12.60 | 5.40 |
| 63 | | >100 | 21.40 |
| 64 | 18.9 | | |
| 65 | 38.4 | | |
| 66 | 52.8 | | |
| 67 | 65.6 | | |
| 68 | 21.1 | | |
| 69 | >100 | | |
| 70 | 29.8 | | |
| 71 | >50 | | |
| 72 | >50 | | |
| 73 | >50 | | |
| 74 | >50 | | |
| 75 | >100 | | |
| 76 | >50 | | |
| 77 | 92.8 | | |
| 78 | 42.4 | | |
| 79 | 64.0 | | |

All publications and patent applications cited herein are incorporated by reference to the same extent as if each individual publication or patent application was specifically and individually indicated to be incorporated by reference.

Although certain embodiments have been described in detail above, those having ordinary skill in the art will clearly understand that many modifications are possible in the embodiments without departing from the teachings thereof. All such modifications are intended to be encompassed within the claims of the invention.

### REFERENCES

1. A Novel and Efficient Approach for the Combinatorial Synthesis of Structurally Diverse Pyrimidines on Solid Support. Obrecht, D.; Abrecht, C.; Grieder, A.; Villalgordo, J. M. Helvetica Chimica Acta, 1997, 80, 65.

2. Preparation of 2-Acylaminopyrimidines and Analogs as Antithrombotics. Lehmann-Lintz, T.; Nar, H.; Wienen, W.; Stassen, J. M. DE 19851421; WO 00/27826.

3. Preparation of Pyrimidine Derivatives as Agrochemical Fungicides and Herbicides. Urushibata, I.; Yoshimura, T.; Deguchi, T.; Yonekura, N.; Sakai, J.; Hayashi, S. WO93/18012.

4. N, N′-Substituted-1,3-diamino-2-hydroxypropane Derivatives. John, V.; Maillard, M.; Jagodzinska, B.; Beck, J. P.; Gailunas, A.; Fang, L.; Sealy, J.; Tenbrink, R.; Freskos, J.; Mickelson, J.; Samala, L.; Hom. R. WO 03/040096 A2.

5. Thrombin Inhibitors. Isaacs, R. C.; Williams, P. D.; Lyle, T. A.; Staas, D. D.; Savage, K. L. WO 02/22584 A1.

6. Preparation and Palladium-catalyzed Arylation of Indolylzinic Halides. Sakamoto, T; Kondo, Y; Takazawa, N; Yamanaka, H. Journal of the Chemical Society, Perkin Transactions 1: Organic and Bio-Organic Chemistry, 1996, (16), 1927.

7. Homolytic Carbon-carbon Bond Formation on Pyrimidine Derivatives. Sakamoto, T; Sakasai, T; Ono, T; Yamanaka, H. Fukusokan Kagaku Toronkai Koen Yoshishu, 12^{th,} 1979, 181.

8. Studies on Pyrimidine Derivatives. XV. Homolytic Acylation and Amidation of Simply Substituted Pyrimidines. Sakamoto, T; Ono, T; Sakasai, T; Yamanaka, H. Chemical & Pharmaceutical Bulletin, 1980, 28(1), 202.

1. A compound of Formula I: 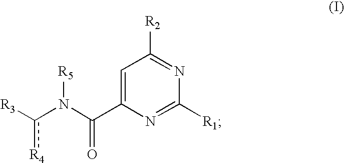
wherein R₁ is halogen, aryl, substituted aryl, heteroaryl, or heterocyclyl, optionally substituted by one or more lower alkyl, aryl or heterocyclyl;
R₂ is lower alkyl, aryl, substituted aryl, heteroaryl, heterocyclyl, or substituted heterocyclyl;
R₃ is hydrogen or carboxy;
R4 is lower alkyl, optionally substituted by aryl, substituted aryl, benzyloxy, or benzylthio; or methylene;
R₅ is hydrogen or lower alkyl;
or an optical isomer, diastereomer or enantiomer thereof; or a pharmaceutically acceptable salt, hydrate, ester or prodrug thereof.

2. A compound of claim 1 wherein R₁ is trifluoromethylphenyl, biphenyl, furyl, thienyl, substituted piperidinyl, or substituted piperazinyl.

3. The compound of claim 1 wherein R₂ is tetrahydroquinolinyl, tetrahydroisoquinolinyl, substituted piperidinyl, or substituted piperazinyl.

4. The compound of claim 1 wherein R₂ is methyl.

5. The compound of claim 1 wherein R₃ is carboxy.

6. The compound of claim 1 wherein R₄ is benzylthiomethyl, benzyloxymethyl, or 4-chlorobenzyl.

7. The compound of claim 1 having the formula:

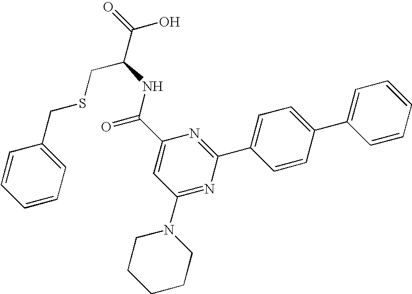

8. A compound of claim 1 having the formula:

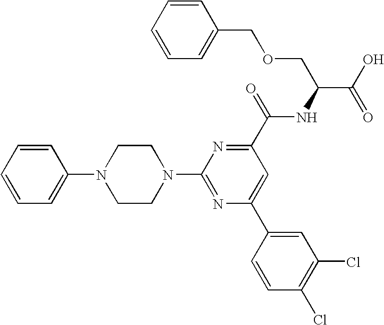

9. A compound of claim 1 having the formula:

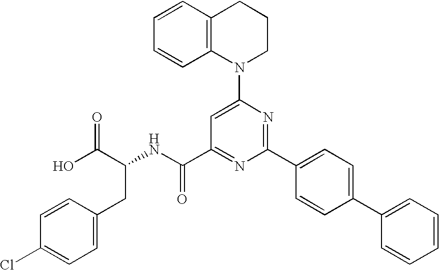

10. A compound of claim 1 having the formula:

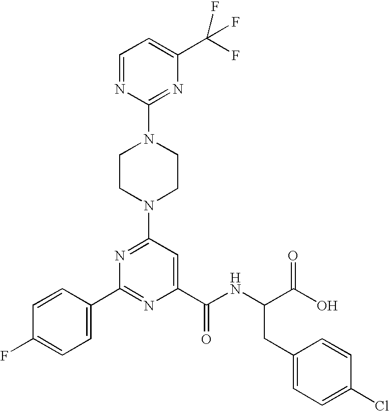

11. A compound of claim 1 having the formula:

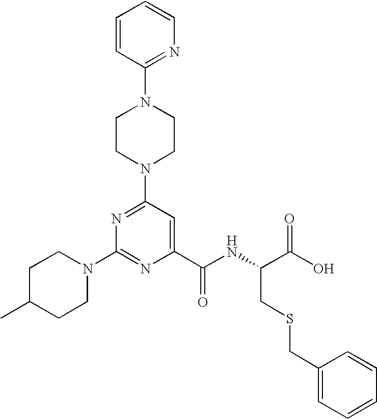

12. A method of inhibiting bacteria with Type III protein secretion systems, said method comprising administration of an effective amount of a compound according to claim 1 to a subject in need of treatment for infection by said bacteria with Type III protein secretion systems.
